(19) United States
(12) Patent Application Publication
(10) Pub. No.: US 2014/0012388 A1
(43) Pub. Date: Jan. 9, 2014

(54) RADIAL HEAD PROSTHESES AND TRIALS

(71) Applicants: Mayo Foundation for Medical Education and Research, Rochester, MN (US); IMDS Corporation, Providence, UT (US)

(72) Inventors: James Brownhill, Orlando, FL (US); Andrew Rynearson, Winter Springs, FL (US); Andrew Palmer, Orlando, FL (US); Michael Chad Hollis, Collierville, TN (US)

(73) Assignees: Mayo Foundation for Medical Education and Research, Rochester, MN (US); IMDS Corporation, Providence, UT (US)

(21) Appl. No.: 13/937,057

(22) Filed: Jul. 8, 2013

Related U.S. Application Data
(60) Provisional application No. 61/668,814, filed on Jul. 6, 2012.
(60) Provisional application No. 61/691,413, filed on Aug. 21, 2012.

(51) Int. Cl. A61F 2/38

(52) U.S. Cl. CPC A61F 2/3804
USPC 623/20.13

(57) ABSTRACT

Radial head prostheses and trialing devices. A radial head prosthesis includes a head component and a stem component, and may include an intermediate component. The head component may be offset from the stem component. The head component includes a bearing surface having an axis of rotation, which may be radially and/or angularly offset from the central longitudinal axis of the stem component. A radial head trialing device may include cutting edges for resection of a distal radius. Another trialing device may allow for adjusting the radial offset and/or angular position of a trial head component from a trial stem component.

### CROSS-REFERENCE TO RELATED APPLICATIONS

This application is a non-provisional of:

pending U.S. Provisional Patent Application No. 61/668,814, filed Jul. 6, 2012, which carries Applicant's docket No. IMDS-1 PROV, and is entitled ADJUSTABLE RADIAL HEAD PROSTHESES; and

pending U.S. Provisional Patent Application No. 61/691,413, filed Aug. 21, 2012, which carries Applicant's docket No. MAYO-1 PROV, and is entitled ADJUSTABLE RADIAL HEAD TRIALS.

The above-identified documents are incorporated herein by reference.

### FIELD OF THE DISCLOSURE

This disclosure relates to prostheses and trialing systems for replacing the radial head. The prostheses may be implanted in the proximal radius to replace a portion of the elbow joint.

### BACKGROUND OF THE INVENTION

Indications for the replacement of the radial head can include: a) degenerative or post-traumatic disabilities presenting pain, crepitation, and decreased motion at the radio humeral and/or proximal radioulnar joint with: joint destruction and/or subluxation visible on ray; and/or resistance to conservative treatment; b) primary replacement after fracture of the radial head; c) symptomatic sequelae after radial head resection; and d) revision following failed radial head arthroplasty, among others.

In radial head replacement procedures, a radial head prosthesis may be implanted into the intramedullary canal of the proximal radius. The radial head prosthesis may cooperate with an ulna or ulnar prosthesis to provide radioulnar joint articulation. The radial head prosthesis may cooperate with a humerus or humeral prosthesis to provide radiohumeral joint articulation.

Typically, in preparation for implantation, the radial head and a portion of the proximal radius is resected to prepare for receiving the prosthesis. It can be difficult to perform a freehand resection of the radius which is oriented normal to the longitudinal axis of the intramedullary canal. It would be desirable to create a device which can perform a resection of the proximal radius which is intrinsically oriented at a selected angle relative to the longitudinal axis of the intramedullary canal. In many cases, radial head replacement is being done on patients whose native anatomy has been degraded or fractured, so it can also be difficult to determine the specific angle at which the radial head should be positioned for proper articulation with the humerus and/or ulna.

Disclosed herein are radial head prostheses which include a stem component, a radial head component, and may also include an intermediate component. The stem component may be shaped to be implanted in the proximal radius, and the radial head component connected to the stem component. The radial head component includes an articular surface for articulation with a natural or prosthetic distal humerus. The position of the radial head relative to the stem may be adjustable; for example the rotation axis of the articular surface may be offset relative to the major axis of the stem component by a distance and/or an angle, providing an eccentric orientation of the head relative to the stem. The radial head prosthesis may be modular, including a set of interchangeable radial head components, a set of interchangeable intermediate components, and/or a set of interchangeable stem components. Practitioners can select and combine the individual components to provide a prosthetic customized for the patient.

The present disclosure also includes devices and methods for bone resection, trial sizing and in situ adjustment of radial head replacements. One example includes devices which can perform both as an implant trial, and as a preparatory instrument for cutting the proximal radius in preparation for implantation of a radial head implant. These devices include a stem component, an intermediate component which may have cutting blades, and variously sized and shaped trial head components. The stem component may be placed in the intramedullary canal of the proximal radius and the intermediate component attached to the stem component. The intermediate component may then be rotated to resect the proximal radius, producing a resection which is normal to the longitudinal axis of the intramedullary canal. Various trial head components may be attached to the intermediate component to determine an optimally sized and shaped radial head prosthesis. In some embodiments the stem may instead be used as the cutting instrument, for instance in designs where the stem has a collar which has cutting features.

Another example includes a trial device which provides distance offset adjustment of a trial radial head component. The device includes a stem component, an intermediate component, and a head component. A spring is positioned between the intermediate component and the head component, and a screw is actuable to move the intermediate component and stem components relative to the head component to adjust the offset of the head component.

Another example includes a trial device which provides angular adjustment of a trial radial head component. The device includes a stem component, an intermediate component, and a head component. The intermediate component is asymmetrically shaped, such that rotation of the intermediate component between the stem component and the trial head component angularly adjusts the orientation of the trial head component relative to the stem component. This allows the practitioner to determine the optimal angulation for the articular surfaces of a radial head prosthesis.

Another example includes a trial device which provides both distance offset adjustment of the radial head component, and angular adjustment of the radial head component. The device includes a stem component, several intermediate components, and a head component. One intermediate component is asymmetrically shaped, such that rotation of the intermediate component between the stem component and the trial head component angularly adjusts the orientation of the trial head component relative to the stem component. Another intermediate component includes a screw which may be actuated to move the intermediate component and the head component relative to the stem component, to adjust the offset of the head component.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1A is a perspective view of a radial head prosthesis having an offset radial head component and a stem component; FIG. 1B is another perspective view of the radial head prosthesis of FIG. 1A; FIG. 1C is a side view of the radial head prosthesis of FIG. 1A;

FIG. 1D is a bottom view of the radial head prosthesis of FIG. 1A;

FIG. 2 is a side cross-sectional exploded view of the radial head prosthesis of FIG. 1A, taken along line A-A in FIG. 1D;

FIG. 3A is a side view of a radial head prosthesis having an angled head component, an intermediate component, and a stem component; FIG. 3B is another side view of the radial head prosthesis of FIG. 3A, rotated 90° from the view of FIG. 3A;

FIG. 4 is an exploded view of the radial head prosthesis of FIG. 3A;

FIG. 5 is a side cross-sectional view of the radial head prosthesis of FIG. 3A, taken along line B-B in FIG. 3A;

FIG. 6 is a side perspective view of another radial head prosthesis having a head component, an intermediate component, and a stem component;

FIG. 7 is an exploded view of the radial head prosthesis of FIG. 6;

FIG. 8 is a side cross-sectional view of the radial head prosthesis of FIG. 6,

FIG. 9A is a side view of a radial head prosthesis having an offset radial head component, an intermediate component, and a stem component; FIG. 9B is a bottom view of the head component of FIG. 9A;

FIG. 10 is an exploded view of the radial head prosthesis of FIG. 9;

FIG. 11A is a side view of another radial head prosthesis having an offset radial head component, an intermediate component, and a stem component; FIG. 11B is a bottom view of the head component of FIG. 11A;

FIG. 12A is a side view of another radial head prosthesis having an offset radial head component, an intermediate component, and a stem component; FIG. 12B is a bottom view of the head component of FIG. 12A;

FIG. 13 is a side view of another radial head prosthesis having a variable offset radial head component;

FIG. 14 is an exploded view of the radial head prosthesis of FIG. 13;

FIG. 15A is a top view of the radial head prosthesis of FIG. 13; FIG. 15B is a bottom view of the radial head prosthesis of FIG. 13;

FIG. 16A is a side view of a radial head trial device including a trial head component, an intermediate component and a stem component; FIG. 16B is a bottom perspective view of the radial head trial device of FIG. 16A;

FIG. 17 is an exploded view of the radial head prosthesis of FIG. 16A;

FIG. 18A is a bottom view of the radial head trial device of FIG. 16A; FIG. 18B is a side cross-sectional view of the radial head trial device of FIG. 16A, taken along line C-C of FIG. 18A;

FIG. 19A is a side view of another radial head trial device including a trial head component, an intermediate component and a stem component; FIG. 19B is another side view of the device of FIG. 19A rotated 90° from the view of FIG. 19A; FIG. 19C is a top perspective view of the device of FIG. 19A; FIG. 19D is a bottom perspective view of the device of FIG. 19A;

FIG. 20 is an exploded view of the radial head prosthesis of FIG. 19A;

FIG. 21A is a cross-sectional view of the trial head component of FIG. 19A taken along line E-E of FIG. 19B; FIG. 21B is a is a cross-sectional view of the trial head component of FIG. 19A taken along line D-D of FIG. 19A;

FIG. 22 is a top view of the stem and intermediate components of the device of FIG. 19A, and a screw and pins of the device;

FIG. 23 is a bottom view of the trial head component and intermediate components of the device of FIG. 19A;

FIG. 24 is a side view of another radial head trial device including a trial head component, an intermediate component and a stem component;

FIG. 25 is an exploded view of the radial head trial device of FIG. 24;

FIG. 26 is a side-cross-sectional view of the radial head trial device of FIG. 24;

FIG. 27 is a perspective view of another radial head trial device including a trial head component, several intermediate components, and a stem component;

FIG. 28 is an exploded view of the radial head trial device of FIG. 27;

FIG. 29A is a side view of a subassembly of the radial head trial device of FIG. 28 showing a mechanism for head offset adjustment; FIG. 29B is a superior view of the subassembly of FIG. 29A;

FIG. 30A is a side view of the radial head trial device of FIG. 28 showing a mechanism for head angular adjustment; and FIG. 30B is a side view of the radial head trial device of FIG. 28 rotated 180° from the view of FIG. 30A.

### DETAILED DESCRIPTION OF THE PREFERRED EMBODIMENTS

The present disclosure relates to radial head implants, radial head trials, and methods for trialing and implantation. Those of skill in the art will recognize that the following description is merely illustrative of the principles of the disclosure, which may be applied in various ways to provide many different alternative embodiments. This description is made for the purpose of illustrating the general principles of this invention and is not meant to limit the inventive concepts in the appended claims.

In this specification, standard medical directional terms are employed with their ordinary and customary meanings. Superior means toward the head. Inferior means away from the head. Anterior means toward the front. Posterior means toward the back. Medial means toward the midline, or plane of bilateral symmetry, of the body. Lateral means away from the midline of the body. Proximal means toward the trunk of the body. Distal means away from the trunk.

In this specification, a standard system of three mutually perpendicular reference planes is employed. A sagittal plane divides a body into bilaterally symmetric right and left portions. A coronal plane divides a body into anterior and posterior portions. A transverse plane divides a body into superior and inferior portions.

Any of the articular or bearing surfaces disclosed herein may replace at least a portion of a natural articular surface of an intact proximal radius, and may articulate directly against a corresponding natural or artificial articular surface.

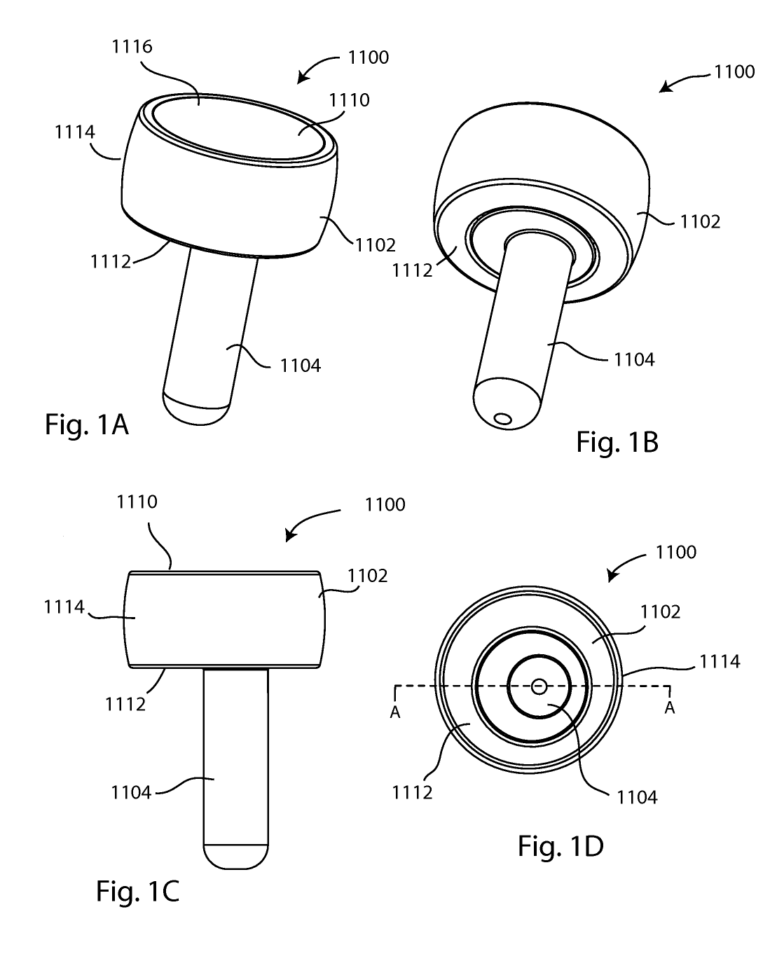

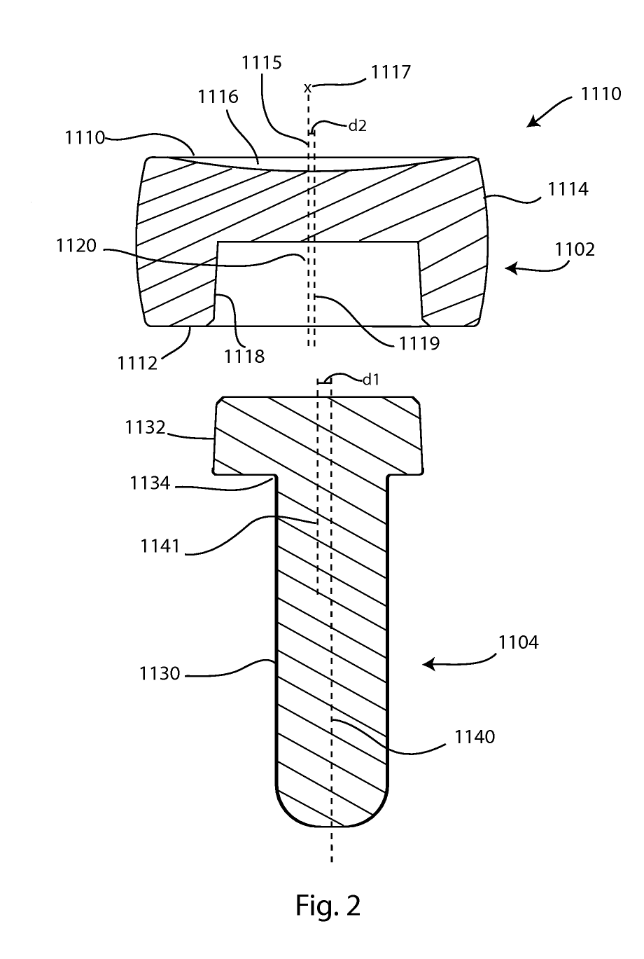

Referring to FIGS. 1A-1D and 2, a radial head prosthesis 1100 includes a head component 1102 and a stem component 1104, which may be referred to as a head and a stem.

Head 1102 includes a first side 1110 opposite a second side 1112. A peripheral wall 1114 extends between the first and second sides 1110, 1112. In the example of FIG. 1, the exterior surface of the peripheral wall 1114 is slightly bowed, convex, or barrel-shaped between the first and second sides 1110, 1112; in other embodiments the exterior surface of the peripheral wall 114 may be cylindrical. The peripheral wall 1114 defines a central longitudinal axis 1115 which extends between the first and second sides 1110, 1112. The peripheral wall 1114 may be a surface of revolution about the central longitudinal axis 1115. A cross section of the peripheral wall 1114, taken perpendicular to the axis 1115, may be circular, oval, rectangular, asymmetric, or polygonal, The peripheral wall 1114 may articulate with a natural or artificial ulnar articular surface. Another articular surface 1116 is on the first side 1110, and may include a dished or concave portion, such as a semi-spherical, elliptical, parabolic, or other surface of revolution. In this specification, a surface of revolution may be described as directional if the surface of revolution has a single axis of rotation, such as surfaces of revolution generated from parabolas or ellipses, for example. In this specification, a surface of revolution may be described as non-directional or omnidirectional if the surface of revolution has an infinite number of axes of revolution, such as surfaces of revolution generated from circles, for example (spherical surfaces). The articular surface 1116 may otherwise be convex or flat. A center point of rotation 1117 of the articular surface 1116 may lie on the central longitudinal axis 1115 as shown, so that the articular surface 1116 is centered on the first side 1110. In another example, the center of rotation 1117 may be offset from the axis 1115 or the center of the first side 1110. The articular surface 1116 may articulate with a natural or artificial humeral articular surface. The second side 1112 includes a head attachment feature 1118, which may be a recess. For example, the head attachment feature 1118 may be a groove across the second side 1112, or a frustoconical socket. The head attachment feature 1118 has a central longitudinal axis 1119 which may extend generally parallel to the central longitudinal axis 1115 of the peripheral wall 1114 as shown; in other examples, the axis 1119 may be coaxial with, intersect, or be skewed to the axis 1115. FIG. 1 illustrates an example in which the axis 1119 is offset from the axis 1115 by a distance d2. The head 1102 has a rotation axis 1120 which may be centered in the articular surface 1116 so that the axis 1120 passes through the center point 1117. In examples where the articular surface 1116 is a surface of revolution, the articular surface 1116 is revolved about the axis 1120 and the center point 1117 lies on the axis 1120. In other examples, the axis 1120 may extend normal to the articular surface 1116 and through the center point 1117. The axis 1120 may be coaxial, parallel, intersecting, or skewed with regard to the axis 1115 and/or the axis 1119. In other words, the axis 1120 may be offset from the axis 1115 by an angle and/or a distance, or coaxial with the axis 1115.

The stem component 1104 includes a stem shaft 1130 and a stem attachment portion 1132. A stem transition area 1134 may lie between the shaft 1130 and the attachment portion 1132. The transition area 1134 may be curved, angled, chamfered, stepped or otherwise shaped to provide a transition between the shaft 1130 and the attachment portion 1132. The stem shaft 1130 and the stem attachment portion 1132 may be integrally formed, or separate parts connected together, for example by a threaded connection, welding, brazing, press fit, taper fit, glue, or other means. In the embodiment depicted, the attachment portion 1132 is larger in outer diameter than the shaft 1130 and overhangs the shaft; in other embodiments the attachment portion may be of equal or smaller diameter than the shaft. The stem attachment portion 1132 may be cylindrical, frustoconical, spherical, ovoid, elliptical, or another shape that is complementary to the head attachment feature 1118. Furthermore, the stem attachment portion 1132 may be an internal feature and the head attachment feature 1118 may be an external feature, contrary to the example shown. The stem attachment feature 1132 is shaped to cooperate with the head attachment feature 1118, providing a connection mechanism which provides a secure fit between the stem 1104 and the head 1102. The connection mechanism may be a Morse taper, press fit, interference fit, snap fit, a meshed connection, an interdigitated connection, a hex connection, a threaded fitting, or another connection known in the art to provide a secure fit between the components. In some examples, the connection may rigidly fix the head 1102 to the stem 1104. The stem shaft 1130 has a longitudinally oriented stem axis 1140. The attachment portion 1132 has a longitudinally oriented central axis 1141. The axis 1141 of the attachment portion 1132 may be offset from the stem axis 1140 by a distance d1 as shown or by an angle; in other embodiments the axis 1141 of the attachment portion 1132 may be coaxial, skew, or angled with respect to the stem axis 1140.

When the head 1102 is coupled to the stem 1104, the head attachment feature 1118 engages the stem attachment feature 1132 and the axes 1119, 1141 are coaxial. In some examples, the head attachment feature 1118 can be coupled to the stem attachment feature 1132 at any one of a plurality of rotational orientations about the coaxial axes 1119, 1141 relative to the stem attachment feature 1132. The connection mechanism may provide discrete rotational orientations, for example if the complementary features 1118, 1132 are polygonal, meshed, interdigitated, splined, ridged and grooved, and the like. In other examples, the connection mechanism may provide an infinite number of rotational orientations, for example if the complementary features 1118, 1132 are round, cylindrical, conical, and the like.

In one example, the head component 1102 can be rotatably adjusted about the coaxial axes 1119, 1141 with respect to the stem component 1104 prior to locking the head 1102 to the stem 1104 such that the rotation axis 1120 is coaxial with the stem axis 1140, or so that the rotation axis 1120 is offset from the stem axis 1140. In other words, head 1102 rotates about axis 1119, which is coaxial with 1141, therefore axis 1120 rotates around 1119 as the head turns. If d1=d2, then in one rotational orientation, axis 1120 can be coaxial with axis 1140. Depending on the positioning of the head component 1102 relative to the stem component 1104, the rotation axis 1120 may be offset by as little as d1−d2 or as much as d1+d2. This offset may be a medial/lateral offset of the head relative to the stem. In this embodiment, the head component may be secured to the stem component at any one of an infinite continuum of orientations or a discrete number of orientations.

Radial head prosthesis 1100 may be assembled during manufacture, intraoperatively on the back table, or in situ. The offset between the rotation axis 1120 and the stem axis 1140 may be determined pre-implantation or during an implantation procedure. During assembly, head 1102 may be rotated relative to stem 1104 until the desired degree of offset is reached, then head 1102 secured to stem 1104. If needed, head 1102 may be removed from stem 1104, the offset adjusted by rotation of head and/or stem, then the head re-secured to the stem. Head 1102 may be attached to stem 1104 before or after stem 1104 is implanted. It is appreciated that the other prostheses disclosed herein may assembled, reassembled, and/or implanted in a similar manner.

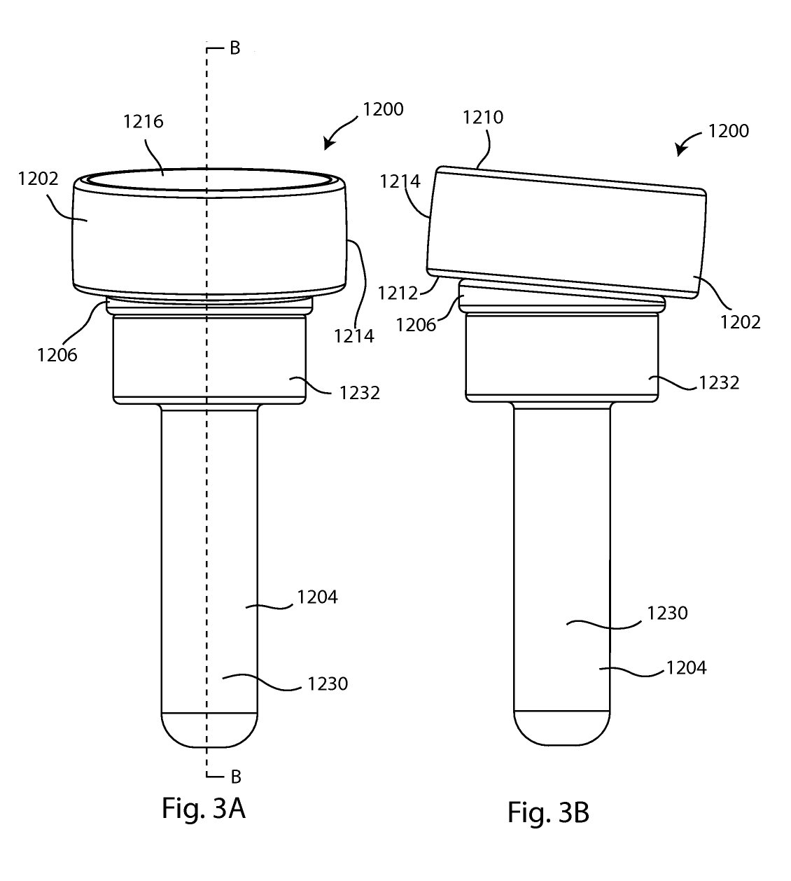

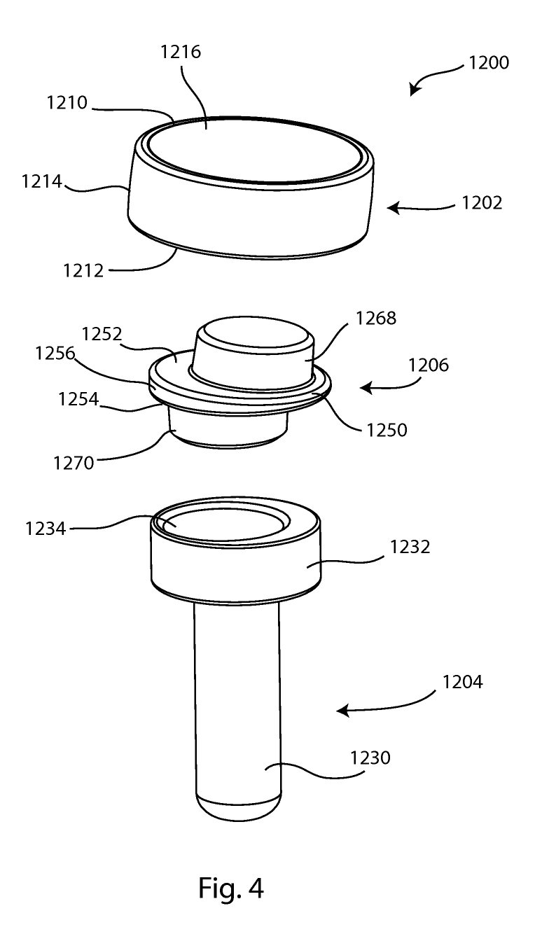

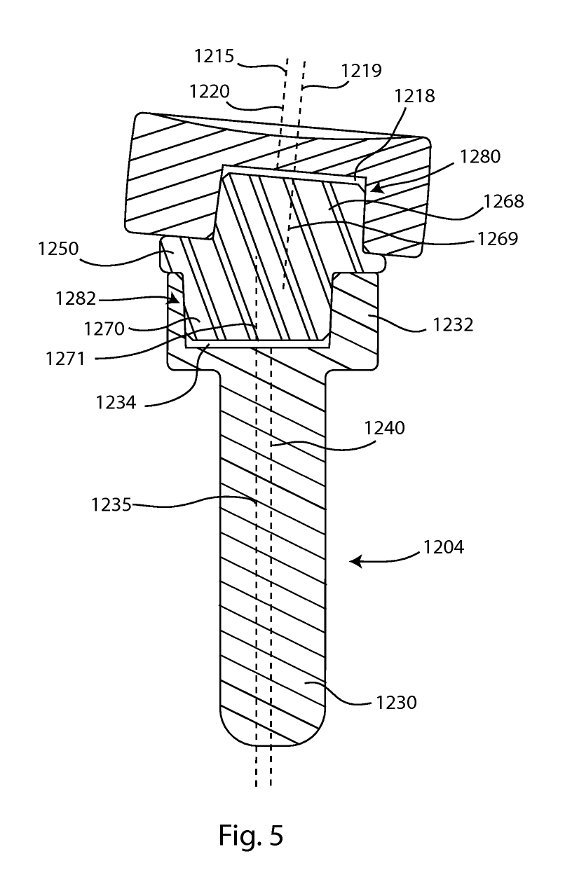

Referring to FIGS. 3A-3B, 4 and 5, another embodiment of a radial head prosthesis is shown. Radial head prosthesis 1200 includes a head component 1202, a stem component 1204, and an intermediate component 1206 which lies between and joins the head and stem components.

The head component 1202 includes a first side 1210 opposite a second side 1212. A peripheral wall 1214 extends between and joins the first and second sides. The peripheral wall) 1214 defines a central longitudinal axis 1215 which extends between the first and second sides 1210, 1212. The first side 1210 includes an articular surface 1216, which may be partially or wholly dished, concave, convex, or may be flat. The articular surface 1216 may be at least a partial surface of revolution, such as a sphere, ellipse, parabola, and the like. The head 1202 includes an attachment feature 1218 for securing the head 1202 to the intermediate component 1206, although in other embodiments the head 1202 may connect directly to the stem component 1204. The attachment feature 1218 has a central longitudinal axis 1219. The articular surface includes a rotation axis 1220 and a center point of rotation 1217 which lies on the axis 1220. In examples where the articular surface 1216 is a surface of revolution, the articular surface 1216 is revolved about the axis 1220. In other examples, the axis 1220 may extend normal to the articular surface 1216 and through the center point 1217. The axis 1220 may be parallel, coaxial, intersecting, or skew relative to the axis 1215. The center point 1217 may lie on the axis 1215, or may be offset from the axis 1215. The axis 1219 may be parallel, coaxial, intersecting, or skew relative to the axis 1215 and/or the axis 1220. From a superior perspective, the head 1202 may be radially symmetrical, while from a medial or lateral perspective it may be asymmetrical; for example the height of the peripheral wall may vary, resulting in a rotation axis 1220 which is not coaxial or parallel with the stem component 1204. Additionally, the rotation axis 1220 may be offset from the geometric center of the first side 1210.

The intermediate component 1206 includes an intermediate body 1250 having a first side 1252, a second side 1254 and a periphery 1264. In some embodiments, the periphery 1264 may vary in thickness, wherein the first side 1252 is angled relative to the second side 1254. In other embodiments, the first side 1252 may be parallel to the second side 1254. The intermediate component may further include a first intermediate attachment feature 1268 and a second intermediate attachment feature 1270. The attachment features 1268, 1270 may be formed as bosses, or protrusions for complementary fit in head and stem sockets or receptacles. In another embodiment, the attachment features 1268, 1270 may be formed as recesses complementarily fitting with bosses or protrusions on the head and stem components. The first intermediate attachment feature 1268 has a central longitudinal axis 1269. The second intermediate attachment feature 1270 has a central longitudinal axis 1271. The axis 1269 may be parallel, coaxial, intersecting, or skew relative to the axis 1271.

The stem component 1204 includes a shaft 1230 and an attachment portion 1232. The shaft 1230 has a longitudinally oriented stem axis 1240. The attachment portion 1232 includes a stem attachment feature 1234 which may be a recess. In another embodiment, attachment feature 1234 may be a boss or another protruding shape which fits complementarily into a recessed attachment feature 1270. The attachment feature 1234 has a central longitudinal axis 1235. The axis 1235 may be parallel, coaxial, intersecting, or skew relative to the axis 1240.

The attachment feature 1218 and first intermediate attachment feature 1268 form a first connection mechanism 1280. The second intermediate attachment feature 1270 and the stem attachment feature 1234 form a second connection mechanism 1282. Although the example shown is a Morse taper, either of both of the first and second connection mechanisms may include a Morse taper, a threaded fitting, a snap fit, a press fit, an interference fit, a meshed connection, an interdigitated connection, a hex connection, and/or a locking connection. The first and second connection mechanisms may be the same or differing types of mechanisms. During assembly of the device, one or both of the first and second connection mechanisms may connect along an assembly axis parallel or coaxial with the longitudinal axis of the stem, or one or both may connect along one or more assembly axes transverse to the longitudinal axis of the stem.

When the head component 1202 is coupled to the intermediate component 1206, the attachment feature 1218 engages the attachment feature 1268 and the axes 1219, 1269 are coaxial. In some examples, the attachment feature 1218 can be coupled to the attachment feature 1268 at any one of a plurality of rotational orientations about the coaxial axes 1219, 1269 relative to the attachment feature 1268. The connection mechanism may provide discrete rotational orientations, for example if the complementary features 1218, 1268 are polygonal, meshed, interdigitated, splined, ridged and grooved, and the like. In other examples, the connection mechanism may provide an infinite number of rotational orientations, for example if the complementary features 1218, 1268 are round, cylindrical, conical, and the like.

When the intermediate component 1206 is coupled to the stem component 1204, the attachment feature 1270 engages the attachment feature 1234 and the axes 1271, 1235 are coaxial. In some examples, the attachment feature 1270 can be coupled to the attachment feature 1234 at any one of a plurality of rotational orientations about the coaxial axes 1271, 1235 relative to the attachment feature 1234. The connection mechanism may provide discrete rotational orientations, for example if the complementary features 1270, 1234 are polygonal, meshed, interdigitated, splined, ridged and grooved, and the like. In other examples, the connection mechanism may provide an infinite number of rotational orientations, for example if the complementary features 1270, 1234 are round, cylindrical, conical, and the like.

The intermediate component 1206 may provide eccentric placement of the head 1202 relative to the stem component 1204. Depending on the positioning of the head 1202 on the intermediate component 1206 and the positioning of the intermediate component 1206 on the stem component 1204, the location of the rotation axis 1220 can vary with respect to the stem axis 1240. This provides discrete or infinite resolution for eccentric offsetting of the rotation axis, allowing intraoperative variability for ideal placement of the articular surface 1216 against the capitellum and the proximal radioulnar joint. This offset may be a medial/lateral offset of the head relative to the stem.

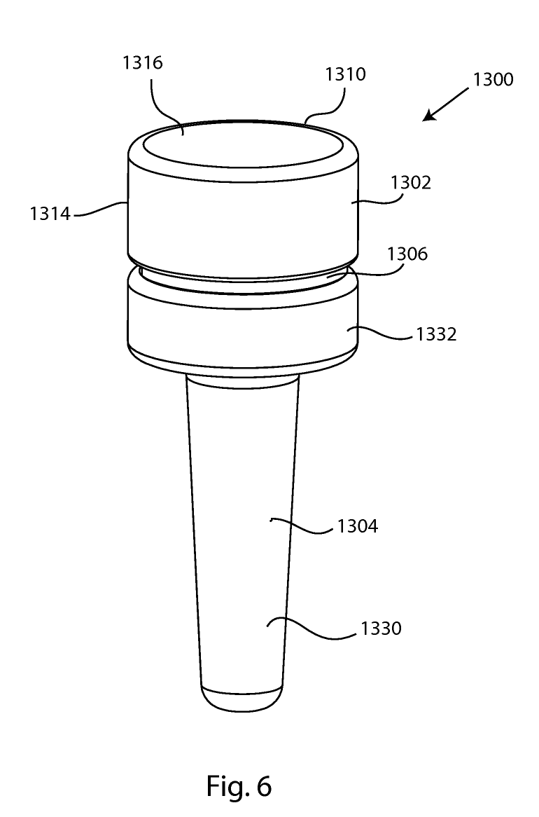

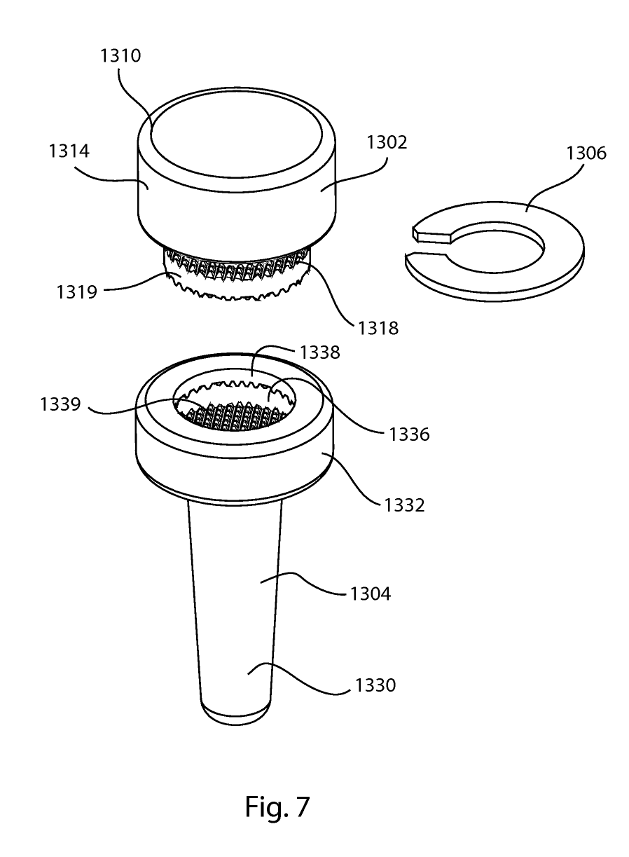

Referring to FIGS. 6-8, another embodiment of a radial head prosthesis is shown. Radial head prosthesis 1300 includes a head component 1302, a stem component 1304 and an intermediate component 1306.

Head component 1302 includes a first side 1310 and a second side 1312 opposite the first side, and a peripheral wall 1314. The peripheral wall 1314 defines a central longitudinal axis 1315 which extends between the first and second sides 1310, 1312. The first side 1310 includes an articular surface 1316, which may be formed as a radial dished shape, such as a semi-spherical, elliptical, parabolic, or other surface of revolution. The articular surface 1316 may be convex or flat in other examples. The articular surface includes a rotation axis 1320 and a center point of rotation 1317. In examples where the articular surface 1316 is a surface of revolution, the articular surface 1316 is revolved about the axis 1320 and the center point 1317 lies on the axis 1320. In other examples, the axis 1320 may extend normal to the articular surface 1316 and through the center point 1317. As with prostheses 1100 and 1200, the thickness of the head may vary, resulting in an angled articular surface 1316 relative to the second side 1312. In the embodiment shown, the rotation axis 1320 is parallel with the axis 1315 of the peripheral wall 1314 as viewed in a longitudinal cross-section; however in other embodiments the rotation axis 1320 may be coaxial, angled, or skew relative to the axis 1315 of the peripheral wall. The center point 1317 may lie on the axis 1315, or may be offset from the axis 1315.

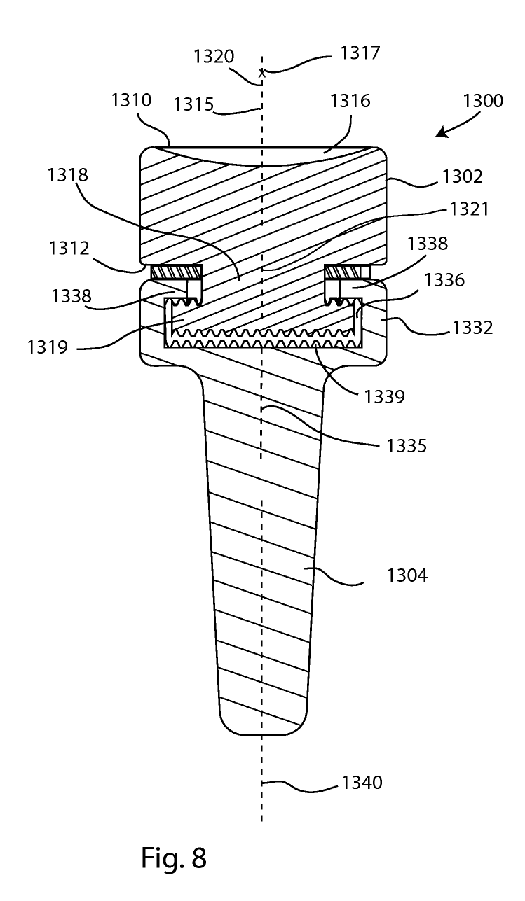

The head component 1302 includes a head attachment feature 1318, which in this embodiment projects from the second side 1312, and is shaped to be received in a complementary recess in the stem component 1304. The head attachment feature 1318 has a central longitudinal axis 1321. The axis 1321 may be parallel, coaxial, intersecting, or skew relative to the axis 1315 and/or the axis 1320. The head attachment feature 1318 may be shaped as a stepped cylinder projecting from the second side 1312. Portions of the attachment feature 1318 may include knurling, splines, or surface roughening. A flange 1319 may be formed on the head attachment feature 1318. The thickness of the flange 1319 may vary so that the flange 1319 appears wedge shaped from the side, or in a cross sectional view such as FIG. 8.

The stem component 1304 includes a shaft 1330 and an attachment portion 1332 at one end of the shaft. The shaft 1330 has a longitudinally oriented stem axis 1340. The attachment portion 1332 includes a shaft attachment feature 1334, in this example shaped as a recess to receive the head attachment feature 1318. The attachment feature 1334 has a central longitudinal axis 1335. The axis 1335 may be parallel, coaxial, intersecting, or skew relative to the axis 1340. Attachment feature 1334 further includes a recess 1336 and a rim 1338 which projects inwardly about the recess. Portions of the rim 1338 and/or recess 1336 may include knurling, splines, or other surface roughening. The thickness of the rim 1338 may vary so that the rim 1338 appears wedge shaped from the side, or in a cross sectional view such as FIG. 8.

The intermediate component 1306 may be inserted between the head component 1302 and the stem component 1304 and may encircle all or a portion of the head attachment feature 1318. The intermediate component 1306 may be shaped as a split ring to allow insertion between the components 1302, 1304 to surround a cylindrical portion of the head attachment feature 1318. When intermediate component 1306 is inserted between the head 1302 and stem 1304 components, the head and stem may be urged toward one another so that the knurled portions mesh together, forming a secure fit between the head and stem. In one embodiment the intermediate component 1306 is inserted between the head second side 1312 and the exterior of the rim 1338, so that the rim 1338 is urged against flange 1319, as illustrated in FIG. 8. In another embodiment the intermediate component 1306 is inserted between the flange 1319 and the rim 1338, so that the flange 1319 is urged against a floor 1339 of the recess 1336. The intermediate component 1306 and rim 1338 cooperate to form a connection mechanism which connects the head component 1302 to the stem component 1304.

When the head component 1302 is coupled to the stem component 1304, the attachment feature 1318 engages the attachment feature 1332. In this embodiment, the axes 1321, 1335 may or may not be coaxial. Diametral clearance between the attachment features 1318, 1332 may exist, and may permit movement of the attachment feature 1318 transverse to the axis 1335. Wedge shaped flange 1319 and/or rim 1338 may cause axis 1321 to intersect or be skew to axis 1335. Furthermore, the attachment feature 1318 may be coupled to the attachment feature 1332 at any one of a plurality of rotational orientations about axis 1321 and/or axis 1335.

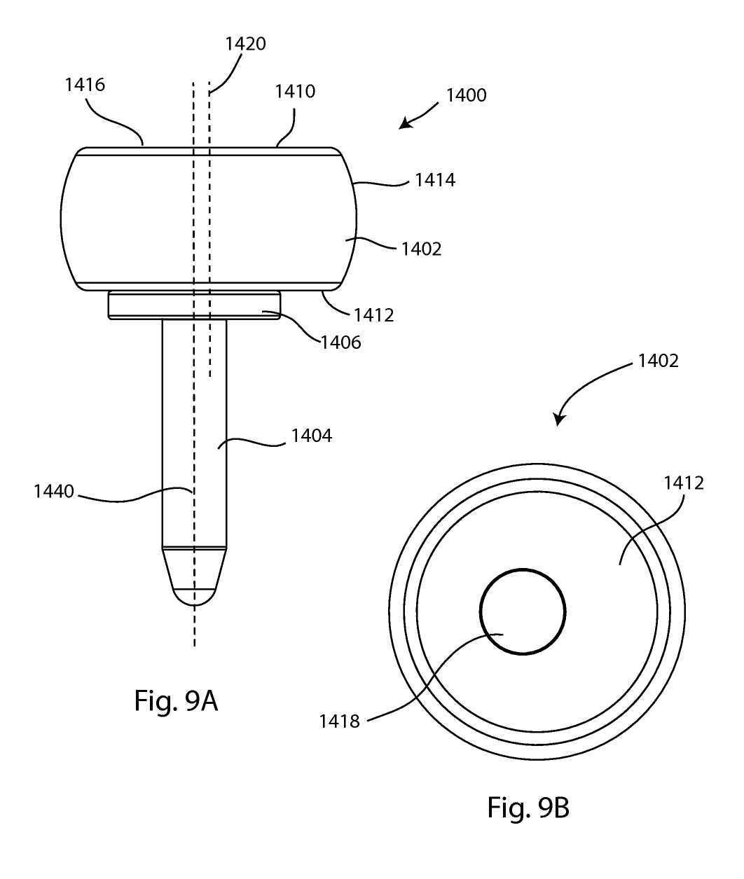

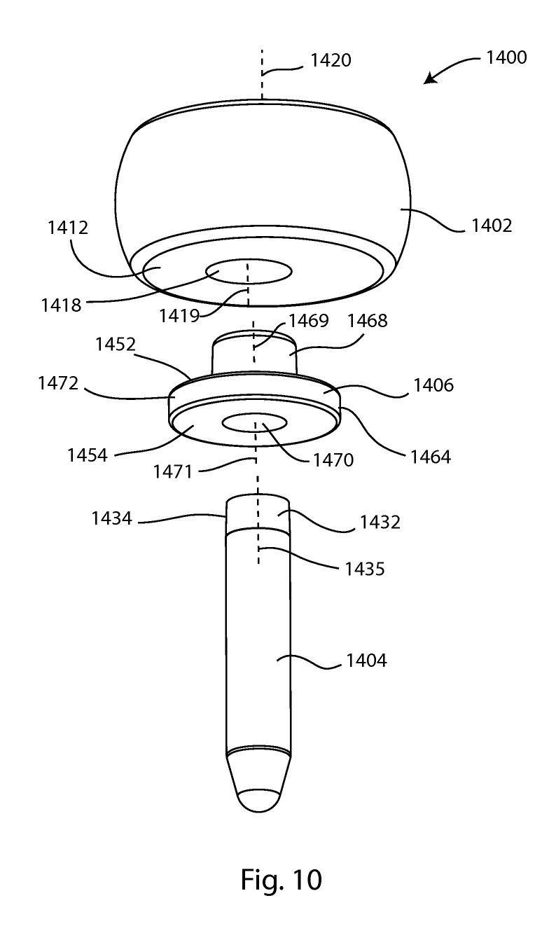

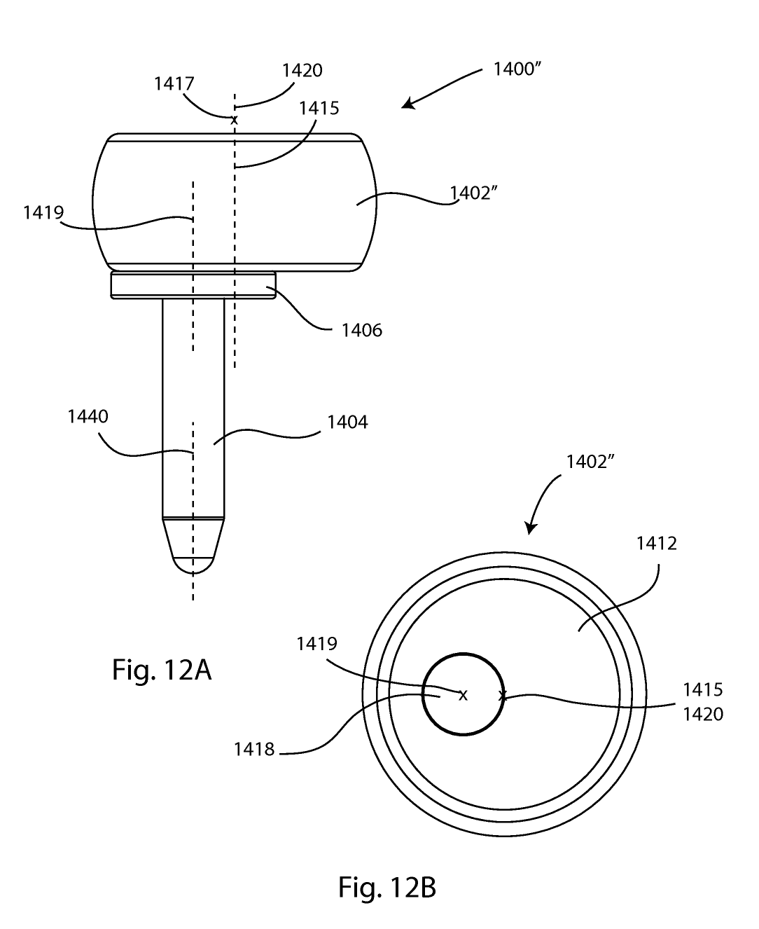

Referring to FIGS. 9A-9B, 10, 11A-11B, and 12A-12B, additional embodiments of radial head prostheses are shown. Radial head prosthesis 1400 includes a head component 1402 and a stem component 1404 joined together by an intermediate component 1406.

The head component 1402 includes a first side 1410 opposite a second side 1412. A peripheral wall 1414 extends between and joins the first and second sides. The peripheral wall 1414 defines a central longitudinal axis 1415 which extends between the first and second sides 1410, 1412. The first side 1410 includes an articular surface 1416, which may be partially or wholly concave, convex, or may be flat. The articular surface 1416 may be a surface of revolution. The head 1402 includes an attachment feature 1418 for securing the head 1402 to the intermediate component 1406, although in other embodiments the head 1402 may connect directly to the stem component 1404. The attachment feature 1418 has a central longitudinal axis 1419. The articular surface of the head component 1402 includes a rotation axis 1420 and a center point of rotation 1417. In examples where the articular surface 1416 is a surface of revolution, the articular surface 1416 is revolved about the axis 1420 and the center point 1417 lies on the axis 1420. In other examples, the axis 1420 may extend normal to the articular surface 1416 and through the center point 1417. The axis 1420 may be parallel, coaxial, intersecting, or skew relative to the axis 1415. The center point 1417 may lie on the axis 1415, or may be offset from the axis 1415. The axis 1419 may be parallel, coaxial, intersecting, or skew relative to the axis 1415 and/or the axis 1420. From a superior perspective, the head 1402 may be radially symmetrical, while from a medial or lateral perspective it may be asymmetrical; for example the height of the peripheral wall may vary, resulting in a rotation axis 1420 which is not coaxial with the stem component 1404. Additionally, the rotation axis 1420 may be offset and/or skewed from the geometric center of the first side 1410. The attachment feature 1418 may also be offset from the geometric center of the second side 1412. It is appreciated that in an alternative embodiment, the views seen in FIGS. 9A, 11A and 12A may include different radial heads 1402, 1402′ and 1402″, each of which have a different offset of the attachment feature 1418 from the geometric center of the second side 1412. Except for this offset, the description of radial head prosthesis 1400 also applies to radial head prostheses 1400′ and 1400″. For example, the stem 1404 and intermediate components 1406 may be the same in all three embodiments.

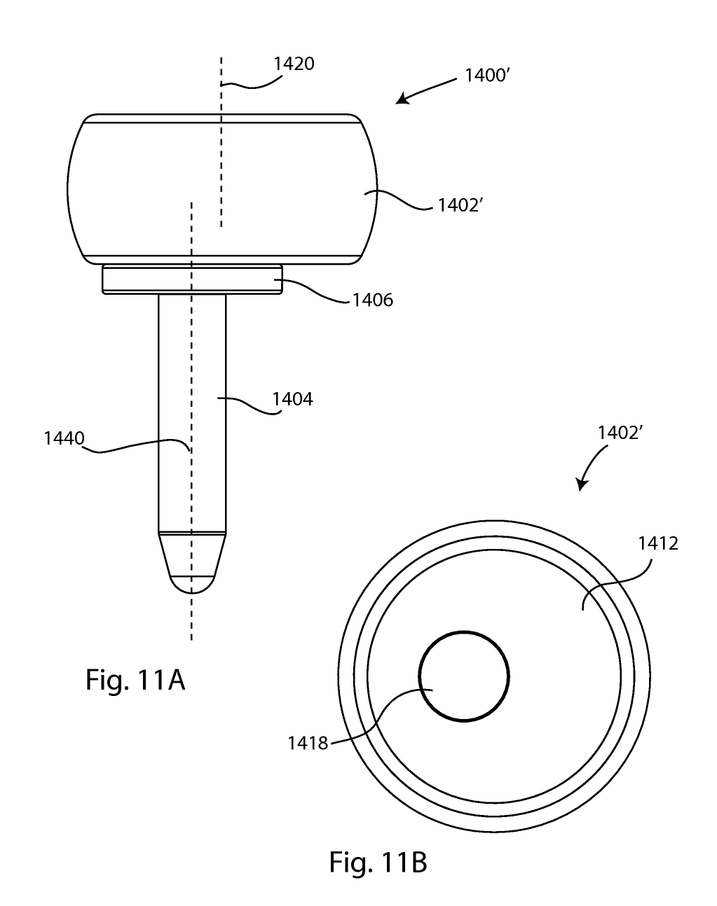

The intermediate component 1406 includes an intermediate body 1450 having a first side 1452, a second side 1454 and a periphery 1464. In some embodiments, the periphery 1464 may vary in thickness, wherein the first side 1452 is angled relative to the second side 1454. The intermediate component may further include a first intermediate attachment feature 1468 and a second intermediate attachment feature 1470. The first intermediate attachment feature 1468 has a central longitudinal axis 1469. The second intermediate attachment feature 1470 has a central longitudinal axis 1471. The axis 1469 may be parallel, coaxial, intersecting, or skew relative to the axis 1471. In the embodiment shown, the first attachment feature 1468 is a protrusion and the second attachment feature 1470 is a recess. The recess may be partially embedded in the protrusion. In the embodiment shown, both the first attachment feature 1468 and second attachment features 1470 are tapered, although in other embodiments they may be straight and/or faceted, or may otherwise share the characteristics of other attachment features disclosed herein. A flange 1472 may project radially from the intermediate component 1406. The flange 1472 may provide a seat for the head component 1402, and may rest upon the prepared radius surface upon implantation. The thickness of the flange 1472 may vary so that the flange 1472 appears wedge shaped from the side, or in a cross sectional view such as FIG. 11A.

The stem component 1404 includes a shaft 1430 and an attachment portion 1432. The attachment portion 1432 includes a stem attachment feature 1434. The shaft 1430 has a longitudinal stem axis 1440. The attachment portion 1432 has a central longitudinal axis 1435.

The attachment feature 1418 and first intermediate attachment feature 1468 form a first connection mechanism 1480. The second intermediate attachment feature 1470 and the stem attachment feature 1434 form a second connection mechanism 1482. Either of both of the first and second connection mechanisms may include a Morse taper, a threaded fitting, a snap fit, a press fit, an interference fit, a meshed connection, an interdigitated connection, a hex connection, and/or a locking connection. The first and second connection mechanisms may be the same or differing types of mechanisms. In other embodiments, the specific shapes of the attachment features forming the connection mechanisms can vary. For example, in another embodiment attachment features 1418, 1434 may be protruding bosses, while attachment features 1468, 1470 are recesses or another complementary shape.

When the head component 1402 is coupled to the intermediate component 1406, the attachment feature 1418 engages the attachment feature 1468 and the axes 1419, 1469 are coaxial. In some examples, the attachment feature 1418 can be coupled to the attachment feature 1468 at any one of a plurality of rotational orientations about the coaxial axes 1419, 1469 relative to the attachment feature 1468. The connection mechanism may provide discrete rotational orientations, for example if the complementary features 1418, 1468 are polygonal, meshed, interdigitated, splined, ridged and grooved, and the like. In other examples, the connection mechanism may provide an infinite number of rotational orientations, for example if the complementary features 1418, 1468 are round, cylindrical, conical, and the like.

When the intermediate component 1406 is coupled to the stem component 1404, the attachment feature 1470 engages the attachment feature 1434 and the axes 1471, 1435 are coaxial. In some examples, the attachment feature 1470 can be coupled to the attachment feature 1434 at any one of a plurality of rotational orientations about the coaxial axes 1471, 1435 relative to the attachment feature 1434. The connection mechanism may provide discrete rotational orientations, for example if the complementary features 1470, 1434 are polygonal, meshed, interdigitated, splined, ridged and grooved, and the like. In other examples, the connection mechanism may provide an infinite number of rotational orientations, for example if the complementary features 1470, 1434 are round, cylindrical, conical, and the like.

During assembly of the device, one or both of the first and second connection mechanisms may connect along an assembly axis parallel or coaxial with the longitudinal axis of the stem, or one or both may connect along one or more assembly axes transverse to the longitudinal axis of the stem. The rotation axis 1420 may be coaxial, parallel, intersecting, skew, or offset from the stem axis 1440. In a method of implantation, the stem 1404 may be implanted into the intramedullary canal or other prepared portion of the radius. The intermediate component 1406 may be joined to the stem 1404 before or after implantation of the stem. The head component may be joined to the intermediate component in situ. As the head component is added, it may be rotated relative to the stem to orient the articular surface 1416 and axis of rotation 1420 at a desired orientation relative to the capitellum. FIGS. 9A, 11A and 12A show heads 1402, 1402′ and 1402″ at different orientations relative to stem 1404 and intermediate component 1406, with varying offset of rotation axis 1420 from stem axis 1440. This offset may be a medial/lateral offset of the head relative to the stem.

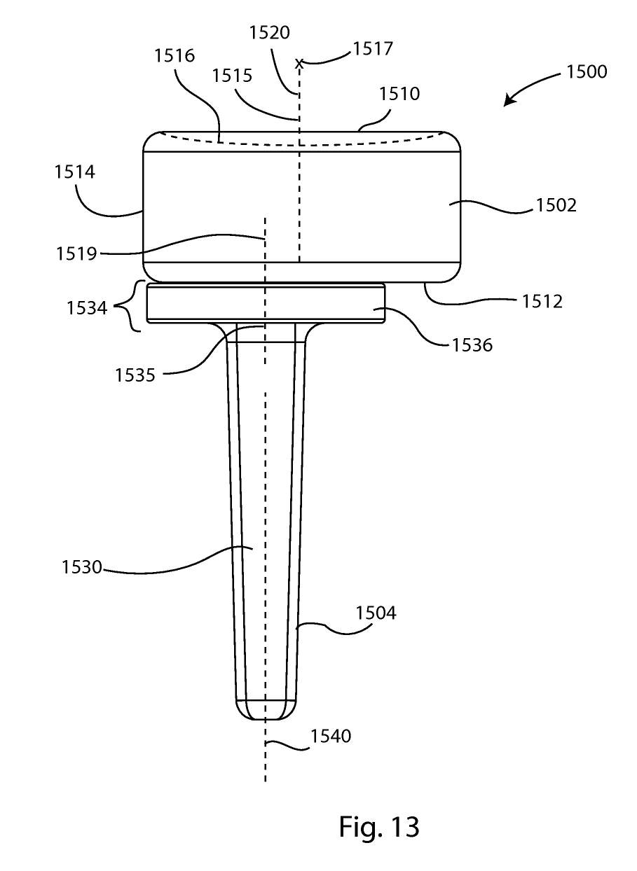

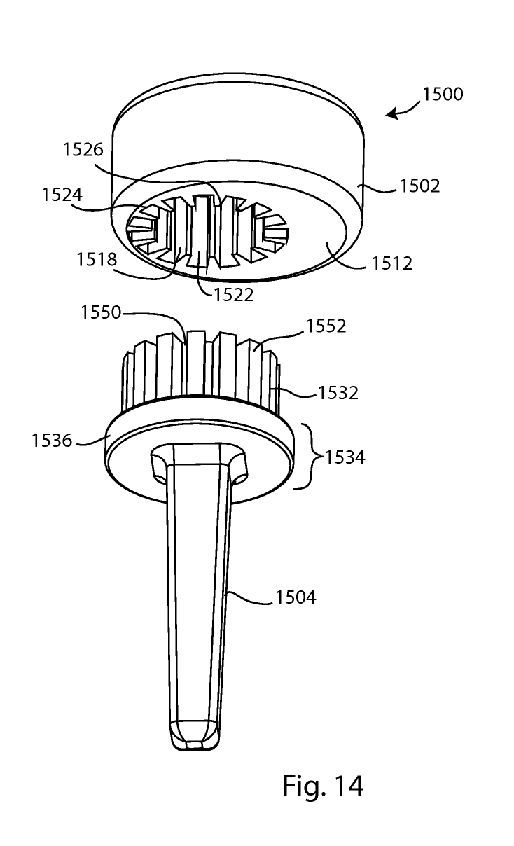

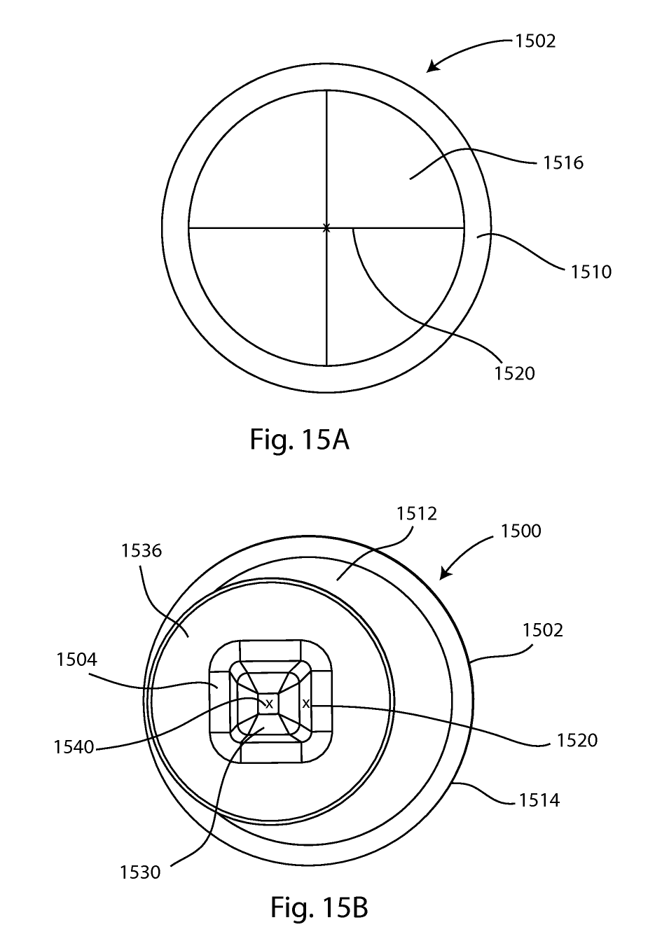

Referring to FIGS. 13, 14, and 15A-15B, another radial head prosthesis is shown. Radial head prosthesis 1500 includes a head component 1502 and a stem component 1504. In the embodiment shown, head 1502 is connected directly with stem 1504; other embodiments may include an intermediate component interposed between the head 1502 and stem 1504.

The head component 1502 is generally disc or puck-shaped, having a first side 1510 opposite a second side 1512. A peripheral wall 1514 extends between and joins the first and second sides. The peripheral wall 1514 defines a central longitudinal axis 1515 which extends between the first and second sides 1510, 1512. The first side 1510 includes an articular surface 1516, which is concave; other embodiments may include partially or wholly concave, convex, or flat articular surfaces. The articular surface 1516 may be a surface of revolution. The head 1502 includes an attachment feature 1518 for securing the head 1502 to the stem component 1504. The attachment feature 1518 has a central longitudinal axis 1519. The attachment feature 1518 is a recess in the second side 1512. The articular surface includes a rotation axis 1520 and a center point of rotation 1517 which lies on the axis 1520. In examples where the articular surface 1516 is a surface of revolution, the articular surface 1516 is revolved about the axis 1520. In other examples, the axis 1520 may extend normal to the articular surface 1516 and through the center point 1517. The axis 1520 may be parallel, coaxial, intersecting, or skew relative to the axis 1515. The center point 1517 may lie on the axis 1515, or may be offset from the axis 1515. The axis 1519 may be parallel, coaxial, intersecting, or skew relative to the axis 1515 and/or the axis 1520. From a superior perspective, the head 1502 is radially symmetrical. However, as in the example shown, the attachment feature 1518 may be offset from the geometric center of the second side 1512, resulting in a rotation axis which is not coaxial with the stem component 1504. Additionally, in some embodiments the rotation axis 1520 may be offset or skewed from the geometric center of the first side 1510.

The stem component 1504 includes a shaft portion 1530, an attachment portion 1532, and a transition portion 1534. The shaft portion may be tapered, and may be received in the intramedullary canal of a prepared radius. The stem includes a center longitudinal axis 1540. The transition portion 1534 in this example is a flange 1536 which projects radially outward farther than either the stem 1530 or the attachment portion 1532; in other embodiments the diameter of the flange may vary or the flange may be omitted. When implanted, the flange 1536 may rest upon the resected radius.

The attachment portion 1532 is radially symmetrical and includes a plurality of discrete teeth 1552 projecting radially outward from an attachment body 1550. The attachment portion 1532 has a center longitudinal axis 1535. The body 1550 and teeth 1552 are shaped to be at least partially received in the attachment feature 1518 of the head component 1502, and may be wholly received therein. The body 1550 and teeth 1552 present a profile which is complementary shaped to mesh with a profile of the attachment feature 1518. The attachment feature 1518 is also radially symmetrical, including a recess 1522 circumscribed by a circular wall 1524 having a plurality of discrete teeth or notches 1526. When the head 1502 is operatively assembled with the stem 1504, the teeth 1552 mesh with notches 1526. The attachment portion 1532 and attachment feature 1518 form a connection mechanism which may be described as a meshed connection. When the head 1502 is coupled to the stem 1504, the attachment feature 1518 engages the attachment portion 1532 and the axes 1519, 1535 are coaxial. It is appreciated that by rotating the head relative to the stem, the head 1502 can be assembled with the stem 1504 at any one of a plurality of discrete orientations. The offset distance between rotation axis 1520 and stem axis 1540 will vary with each discrete locational placement of the head 1502 upon the stem 1504.

Any of the radial head components disclosed herein, including head components 1102, 1202, 1302, 1402, 1402′, 1402″, 1502, and their equivalents may comprise one or materials chosen from a group including stainless steel, cobalt-chrome or its alloys, or titanium and its alloys. In some embodiments, the head articular surface may be a polished articular surface. In some embodiments, the head articular surface may include a polyethylene insert in a metal sleeve.

A flange similar to flange 1472 may be incorporated into any of the embodiments disclosed herein, on a head component, intermediate component, or stem component; the flange intended to rest on the resected proximal radial surface.

Some embodiments may include a stem component 1104, 1204, 1304, 1404 or 1504 having a shaft that is curved, or a shaft that is multi-segmented, having 2 or more angled segments. The outer peripheral surface of the shaft may be generally rounded or cylindrical, may be multi-faceted. In cross-section, the shaft may be circular, square, rectangular, triangular, hexagonal, star-shaped, or another cross-sectional shape.

Any of the stem components disclosed herein, including stem components 1104, 1204, 1304, 1404, and 1504 may be implanted into a prepared bone. The connection between the stem component and the bone may be a press fit or a loose fit, and may incorporate cement, bone ingrowth, or bone ongrowth. The stem may include porous treatments, surface roughening, and/or treatments to encourage bone ingrowth or bone ongrowth. Attachment of the head and/or intermediate component to the stem may occur before or after implantation of the stem.

Any of the embodiments of radial head prostheses described herein may be provided as a modular prosthesis, with separate and interchangeable heads, stems, and intermediate components. Each component may vary in size, to allow assembly of a customized prosthesis. The head components may vary in thickness; and/or shape or location of the articular surface; and/or location or orientation of the axis of rotation. Some embodiments may include an axis of rotation coaxial with the stem axis, while others provide an offset, eccentric and/or angled axis of rotation, relative to the stem axis.

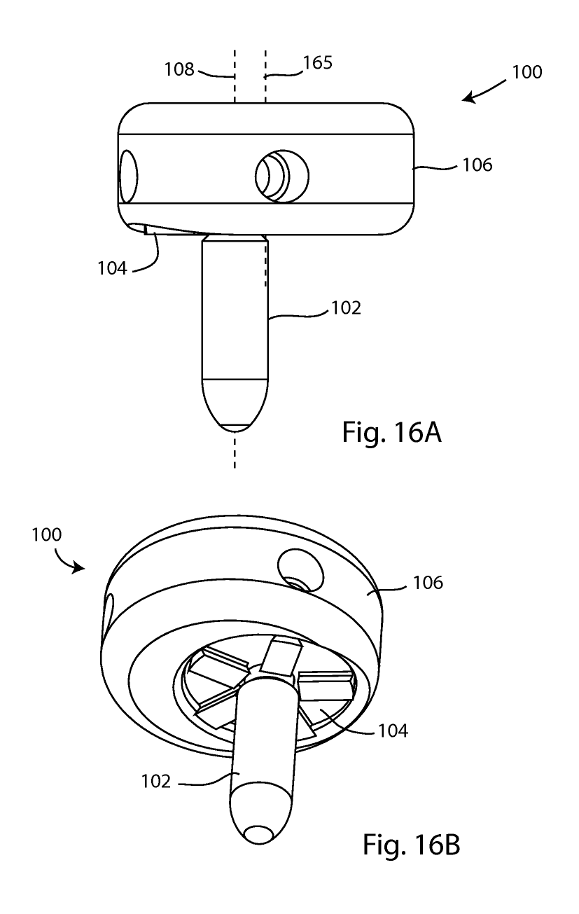

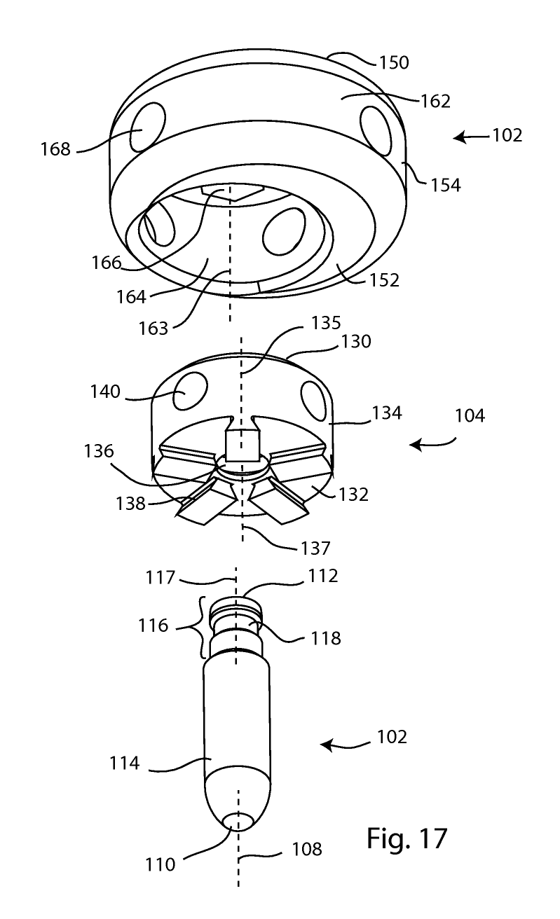

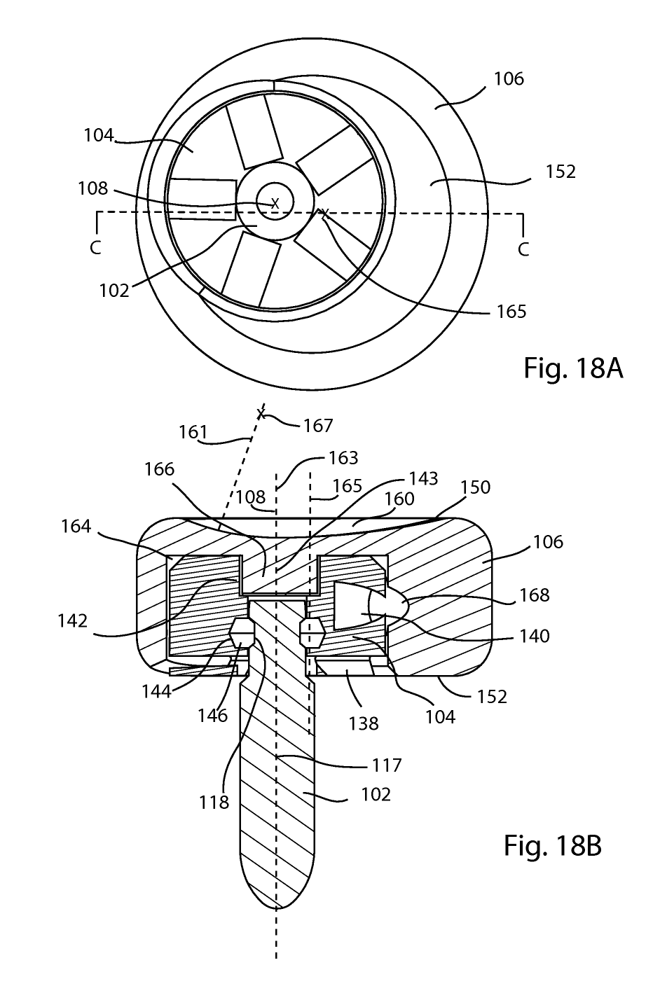

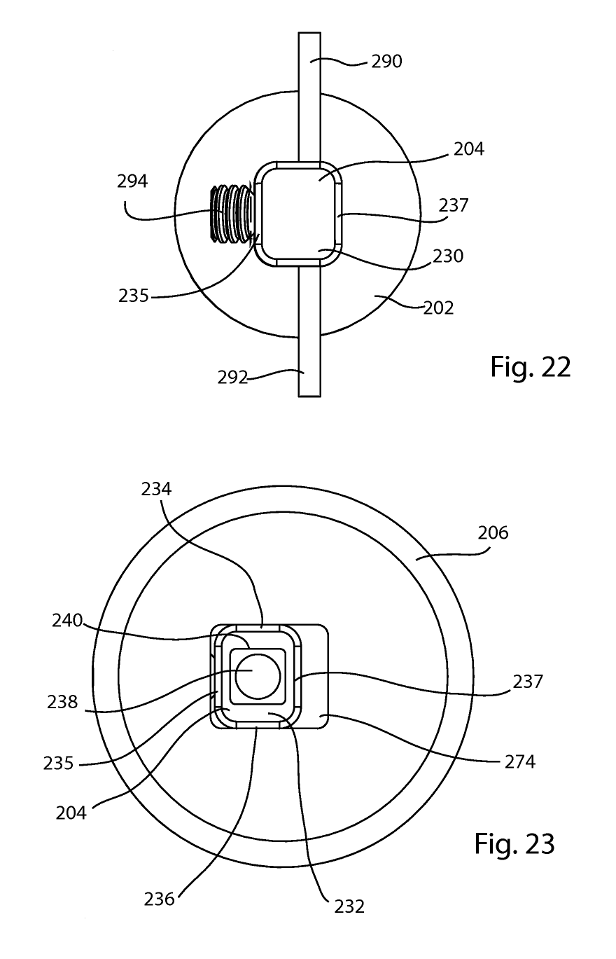

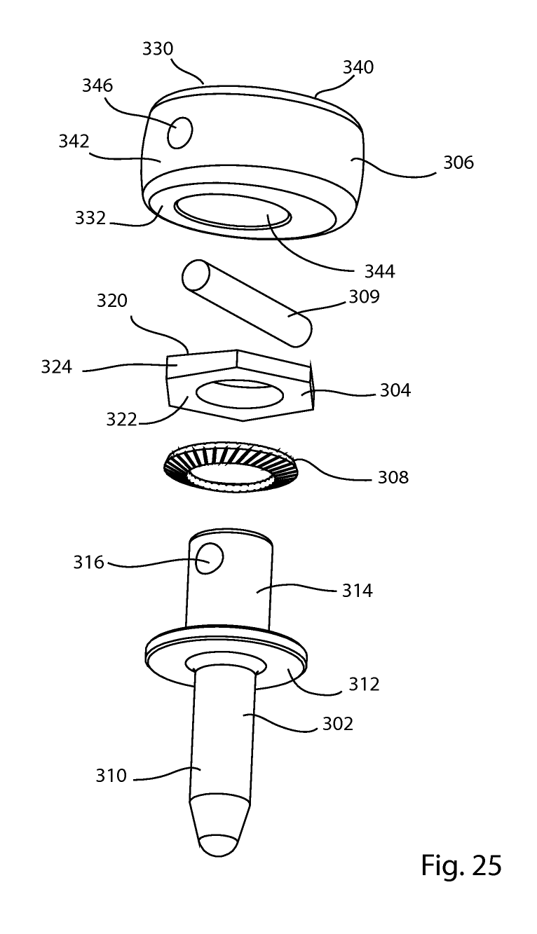

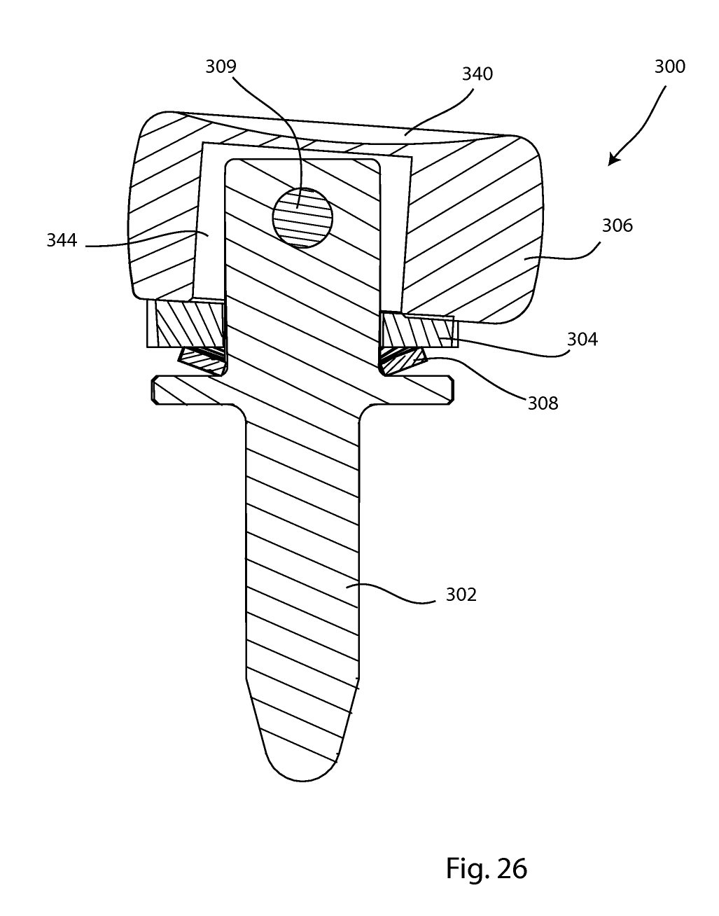

FIGS. 16A-30B and the accompanying description disclose devices and methods which may be used to prepare a distal radial for a radial head implant, including those described above.

Referring to FIGS. 16A-16B, 17, and 18A-18B, a radial head trial device 100 includes a stem component 102, an intermediate component 104, and a head or cap component 106. The stem component 102 and intermediate component 104 may be radially symmetrical about a center longitudinal axis 108 defined by a stem shaft portion 114 of the stem component.

The stem component 102 has a distal end 110 and a proximal end 112. The stem component includes the stem shaft portion 114 and a stem attachment portion 116. The stem attachment portion 116 has a central longitudinal axis 117, which in this example is coaxial with axis 108. A stem groove 118 may be formed in the stem attachment portion 116. The stem component 102 is generally cylindrical, which may allow easy insertion, rotation and removal of the stem component in the intramedullary canal. A variety of different diameter and/or length stem components 102 may be provided. Radial head trial device 100, or subassemblies thereof, may also be described as a resection device.

The intermediate component 104 is generally cylindrical and/or donut-shaped, and includes a superior end 130, an inferior end 132 and a peripheral wall 134 extending between the ends 130, 132. The peripheral wall 134 has a central longitudinal axis 135. The intermediate component may also be referred to as a cutting head or resection component, although in other embodiments the stem component may be the cutting or resection component. A central bore 136 extends between the superior end 130 and the inferior end 132, although in other embodiments the central bore may be blind from either or both ends, not extending completely through the component. The central bore 136 has a central longitudinal axis 137, which in this example is coaxial with axis 135. Several cutting edges or blades 138 are formed on the inferior end 132, creating a cutting surface. In the embodiment shown, the blades extend radially outward from the bore 136 toward the peripheral wall 134, and the cutting edges 138 are perpendicular to the axis 137. Other embodiments may include various numbers and distribution of blades or cutting edges. Several openings 140 extend into the intermediate component 104 from the peripheral wall. The openings 140 are generally transverse to the axis 137, and are sized to receive an instrument or rod which may be inserted into an opening 140 to rotate the intermediate component. In the example shown, the openings 140 are spaced at 90° from one another; other embodiments may include different opening spacings. The bore 136 may terminate superiorly in a shaped recess 142. The shaped recess 142 has a central longitudinal axis 143, which in this example is coaxial with axis 135. A groove 144 may also be formed into the bore 136. The groove 144 in this example is adjacent to the shaped recess 142.

When the intermediate component 104 is placed on the stem 102 with the stem attachment portion 116 fitting into the bore 136, axis 135 is coaxial with axis 108. The groove 144 may be opposite the stem groove 118 such that a donut-shaped gap 146 is formed between the stem 102 and the intermediate component 104. Grooves 144, 118 may be chamfered. A coil spring (not shown) such as a toroidal Bal-Seal™ canted coil spring may be positioned in the gap 146. An O-ring or other retaining ring may be used instead. The attachment portion 116, bore 136, grooves 144 and 118, gap 146, and spring form a connection mechanism. Thus connected, the intermediate component 104 can rotate freely about the center longitudinal axis 108 with no translation along the axis 108. Other connection mechanisms are contemplated to attach the intermediate component to the stem, including a press fit, interference connection, snap fit, a complementary pin and recess, ball plunger, or any other temporary connection that allows the intermediate component to rotate freely about the center longitudinal axis 108 with no axial translation along the axis 108. In some embodiments, the stem 102 and intermediate components 104 may be integrally formed as a single component. In some embodiments, the blades, cutting edges or cutting surface may be removable and/or replaceable. In some embodiments, the blades, cutting edges or cutting surface may be at a non-perpendicular angle relative to the center longitudinal axis 108, in other words, an angle greater than zero and less than ninety degrees. In some embodiments, the blades, cutting edges or cutting surface may be curved to provide a domed or dished resection surface. In some embodiments, the blades, cutting edges or cutting surface may be discontinuous to create ridges, grooves or other surface features in the resected bone surface.

The cap 106 may be generally disc or puck shaped, and includes a superior end 150, an inferior end 152, a cap center axis 165, and a peripheral wall 154 extending between the ends 150, 152. The peripheral wall 154 defines the cap center axis 165, which may be referred to as a central longitudinal axis which extends between the superior and inferior ends 150, 152. A first bearing surface 160, which may be dished, concave, convex, or flat, is formed on the superior end 150 and is shaped to articulate with a distal humerus or distal humerus prosthesis. The first bearing surface 160 may be a surface of revolution. The first bearing surface 160 has a rotation axis 161 and a center point of rotation 167 which lies on the axis 161. In examples where the first bearing surface 160 is a surface of revolution, the first bearing surface 160 is revolved about the axis 161. In other examples, the axis 161 may extend normal to the first bearing surface 160. Axis 161 may be parallel, coaxial, intersecting, or skew relative to axis 165. In the example shown, the center 167 of the first bearing surface 160 is offset from the axis 165, but in other embodiments, the center 167 may be located on the axis 165. A second bearing surface 162 is formed on the peripheral wall 154 and may be shaped to articulate with a proximal ulna or proximal ulna prosthesis. The second bearing surface 162 may be shaped as a cylinder or a continuous loop. A recess 164 extends inwardly from the inferior end 152, the recess sized and shaped to receive the intermediate component 104. The recess 164 has a central longitudinal axis 163. The axis 163 of the recess 164 may be offset from, and parallel to, the cap center axis 165 as in the example shown, resulting in an asymmetrical cap; in other embodiments axis 163 may be centered or coaxial with axis 165, resulting in a radially symmetrical cap with no offset. In other examples, axis 163 may intersect or be skewed relative to axis 165. The overall offset may include an anterior/posterior offset from the cap center axis 165, or a medial/lateral offset, or both. When the cap is coupled to the intermediate component 104 and the stem 102 with the peripheral wall 134 fitting into the recess 164, axis 163 is coaxial with axis 108. Center axis 165 may be parallel with axis 108, or it may be coaxial, intersecting, or skewed relative to axis 108. A boss 166 projects inferiorly into the recess 164. The outer shape of the boss 166 is shaped to complementarily fit the shaped recess 142 at the superior end of the intermediate component bore 136. In the example shown, the boss 166 is a hex shape and shaped recess 142 is likewise hex shaped, although other examples may include other complementary shapes resulting in a keyed fit between the cap and the intermediate component. The cap 102 may further include cap openings 168 which open transversely through the peripheral wall 154 and into the recess 164. When the cap 102 is placed on the intermediate component 104 in a selected orientation, with the boss 166 fitting into the shaped recess 142, the cap openings 168 are coaxial with the intermediate component openings 140. Due to the offset location of recess 164 relative to the peripheral wall 154 of the cap, the cap openings 168 may not be distributed evenly about the cap perimeter.

In a method of use, stem component 102 may be inserted into the intramedullary canal of a proximal radius. The intermediate component 104 may be attached to the stem component 102 before or after insertion of the stem component 102. The subassembly of the stem and intermediate component 102, 104 is rotated so that the blades 138 contact and shave off portions of the proximal radius. Alternatively, the intermediate component 104 may be rotated about the stem 102. An instrument or stylus (not shown) may be inserted into one or more of the openings 140 and/or shaped recess 142 to rotate the components; or the components may be manually rotated. The cap component 106 is attached to the intermediate component and aligned with the distal humerus and/or proximal ulna to determine and select the optimal sized and shaped radial head prosthesis, and/or determine the optimal offset for the head prosthesis. Cap components of various sizes and offsets may be attached and detached to make the determination. Alternatively, the cap component 106 may be attached to the intermediate component 104 before the rotation step, and the entire device 100 may be rotated to resect the proximal radius. When resection and prosthesis selection are complete, the device 100 may be removed and a similarly-dimensioned radial heal prosthesis implanted into the resected radius.

Any of the intermediate components disclosed herein, including intermediate component 104, may comprise ceramic, stainless steel, surgical steel or other suitable metals or alloys. The cutting edges or blades may comprise stainless steel, titanium, cobalt-chrome, ceramic, or other materials suitable for holding a sharp edge. Coatings and/or surface treatments may be used on the cutting edges or blades to enhance performance. The cap components may comprise one or more materials, including but not limited to, polymers including acetal, polyphenylsulfone (PPSU), polyetherimide (PEI), polyether ether ketone (PEEK), polyethylene (PE) including high molecular weight grades; metals including stainless steel, cobalt-chrome or its alloys, or titanium and its alloys; ceramics; or any other articular bearing material.

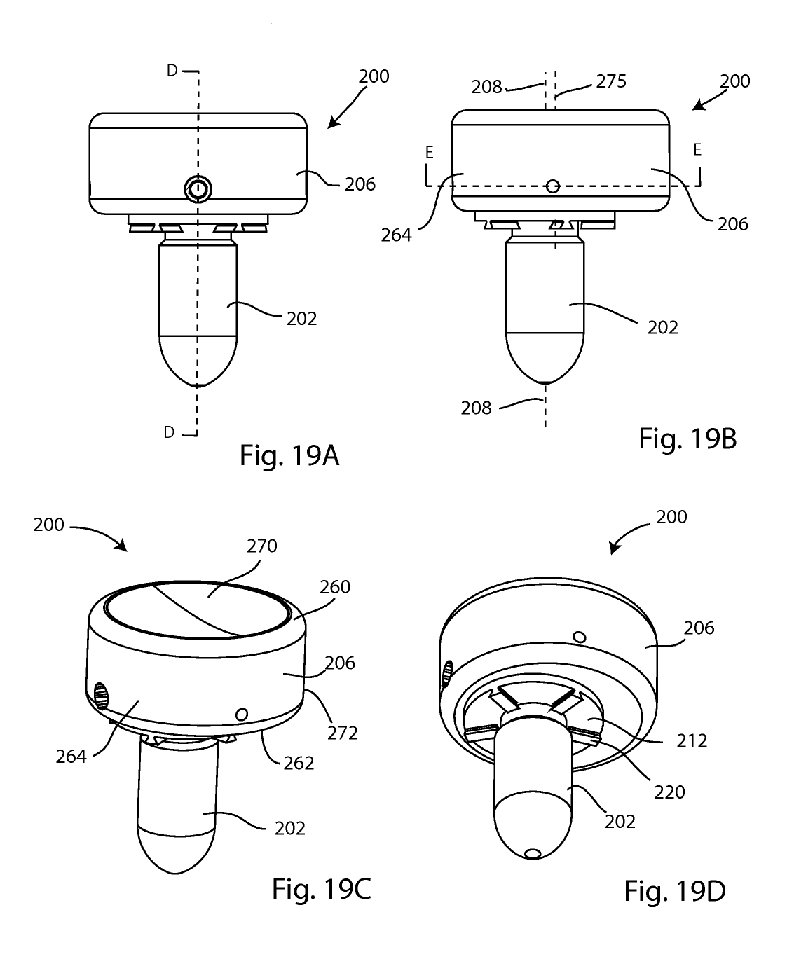

FIGS. 19A-23 show another embodiment of a radial head trial device which may be used to resect a proximal radius and/or to adjust of the offset of the radial head trial relative to the stem. The device 200 includes a stem 202, a head 206, a slider 204, a spring 296, and a set screw 294. The stem is inserted into the proximal opening of the radius after the native radial head has been removed and the proximal shaft has been progressively reamed. The stem 202 is made available in a range of diameters to suit different size patients. The undersurface of the collar on the stem has cutting features which are intended to allow a surgeon to shave any high points on the proximal surface of the radius created during the head resection. The proximal end of the stem has a connection feature (in this embodiment it is a Bal-Seal™ canted coil spring, but other connection features are contemplated) which constrains the stem to the sliding component axially and superiorly-inferiorly. The slider component is also connected to the head component with pins. The pins allow the slider to move in one direction only and prevent the head from disassociating from the slider. Motion of the slider is controlled by the set screw (which is threaded into one side of the head). The spring is located opposite the set screw and functions to keep the slider against the set screw. A scale can be included on the bottom of the head to show the offset of the head from the stem after trialing is complete. The head component can be made in different sizes. The screw hole also functions as a connection for inserting/removing the trial and for rotating the head on the stem to use the cutting fins on the collar of the stem. The cutting surface of the collar removes the need to recut the radius with a saw. The offset adjustment allows the head to be translated in situ with good tactile feedback for the effects on the proximal radio-ulnar joint and the radiocapitellar joint. In situ adjustment may reduce total surgery time because the surgeon is not required to remove the trial and assemble alternative trial configurations. It also provides better comparison between offsets and allows for an infinite resolution of offsets when compared to non-adjustable trials. It also focuses the surgeon's attention on the functional effects of various offsets, rather than on the mathematical progression of offsets in the system. Having one trial for each head size which covers the entire range of offsets may also reduce the inventory of the instrument system.

Radial head trial device 200 includes the stem component 202, the slider or intermediate component 204, and the head or cap component 206. In this embodiment, the cutting surface is integral to the stem component 202. The intermediate component 204 may be rigidly attached to the stem component 204 to form a stem assembly. The cap component 204 may be offset from the stem component 202 as described previously, with a cap center axis not coaxial with the stem center axis. The cap component is translatable relative to the stem assembly to adjust the offset of the cap component.

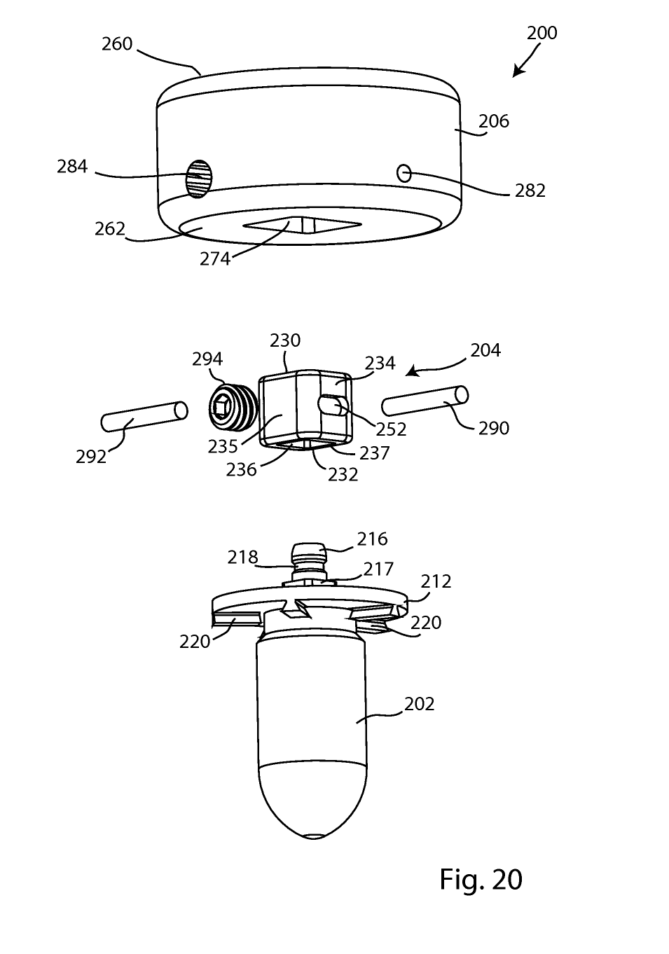

Referring to FIG. 20, an exploded view of device 200 is shown. Stem component 202 has a stem center axis 208, and includes a shaft portion 210, a collar portion 212, and a stem attachment portion 216. The stem attachment portion 216 includes a key 217 which is complementarily shaped to rigidly connect with the intermediate component 204. A stem groove 218 may be formed in the stem attachment portion 216. The shaft portion 210 is generally cylindrical, which may allow easy insertion, rotation and removal of the stem component in the intramedullary canal. A variety of different diameter and/or length stem components 202 may be provided. Several cutting edges or blades 220 are formed on the collar 212, creating a cutting surface. In the embodiment shown, the blades extend radially outward from center of the stem toward the outer edge of the collar 212. Other embodiments may include various numbers and distribution of blades or cutting edges.

The intermediate component 204 may be generally shaped as a rectangular box, having a superior end 230 which may be solid, an inferior end 232 and four sides 234, 235, 236, 237. The interior of the intermediate component 204 comprises a chamber 238. A shoulder 240 is formed around the periphery of the chamber 238 toward the inferior end 232, the shoulder 240 complementarily fitting with the key 217 of the stem component 202 to provide a rigid connection between the intermediate and stem components 204, 202. A groove 242 may also be formed in the periphery of the chamber 238. When intermediate component 204 is fitted onto stem component 202, groove 242 may face stem groove 218 to form a gap for a connection feature (not shown) such as the Bal-Seal™ spring described for device 100. Slots 252, 254 are formed in sides 234 and 236, respectively.

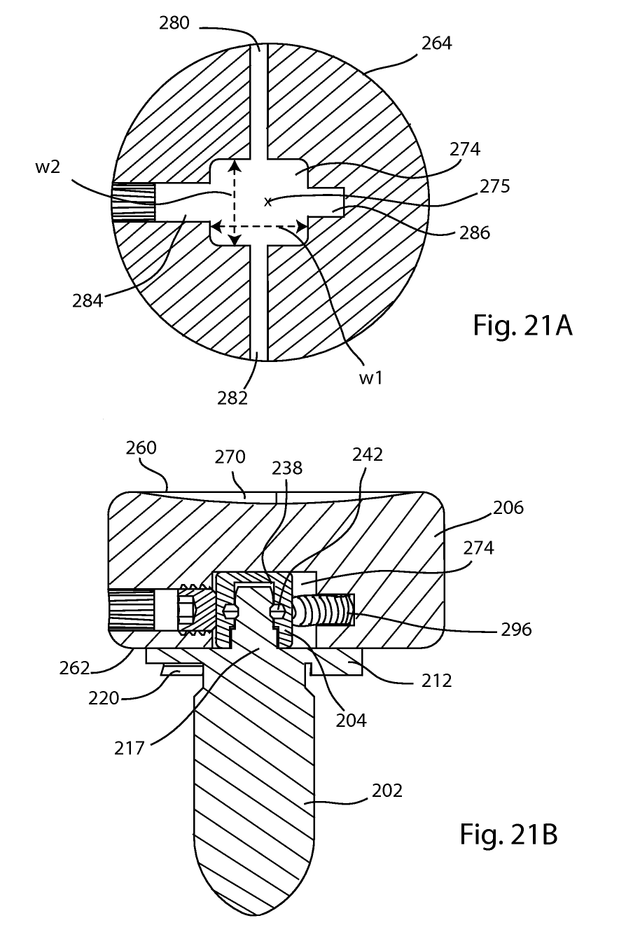

The cap 206 may be disc or puck shaped, and includes a superior end 260, an inferior end 262, and a peripheral wall 264 extending between the ends 260, 262. A first bearing surface 270, which may be dished, is formed on the superior end 260 and is shaped to articulate with a distal humerus or distal humerus prosthesis. In the example shown, the center of the first bearing surface is offset from the center longitudinal axis 208, but in other embodiments may be located on the center longitudinal axis 208. A second bearing surface 272 is formed on the peripheral wall 264 and may be shaped to articulate with a proximal ulna. A recess 274 extends inwardly from the inferior end 262, the recess sized and shaped to receive the intermediate component 204. A width w1 of the recess 274 is greater than the width of the intermediate component measured from side 235 to 237, so that cap component 206 may translate relative to intermediate component 204. The recess 274 may be offset from a cap center axis 275 shown in FIG. 21A. The cap component 206 further includes pin channels 280, 282 which extend from the recess 274 through the peripheral wall 264. The pin channels 280, 282 are sized and shaped to receive pins 290, 292 which extend through slots 252, 254 to tether the intermediate component 204 to the cap component 206 within the recess 274, but allow translation of the intermediate component 204 within the recess 274. A screw channel 284 extends from the peripheral wall 264 into the recess 274. Opposite the screw channel 284, a spring alcove 286 extends out of the recess 274 into the body of the cap component 206. The radial head trial device 200 may further include a screw 294 and a spring 296.

When device 200 is fully assembled, intermediate component 204 fits on stem component 202, with stem attachment portion 216 forming a keyed fitting with complementarily shaped shoulder 204. Cap component 206 fits over intermediate component 204, with the intermediate component received in recess 274, and spring 296 captured in spring alcove 286. Pins 290, 292 are received in channels 280, 282 and extend into slots 252, 254. The screw 294 may be advanced through screw channel 284 to push the slider or intermediate component 204 within recess 274 against the resistance of spring 296, thus moving attached cap component 206 relative to stem component 202. This movement changes the distance between the center longitudinal axis 208 of the stem component and the center axis 275 of the cap component, thus allowing adjustment of the offset of cap component 206 relative to the stem component 202. Device 200 may be inserted into an intramedullary canal of a proximal radius, and rotated to shave off bone tissue until a desired resected surface is obtained. Screw 294 may be advanced and/or retracted to translate cap component 206 until a desired offset is reached which allows proper articulation between the cap bearing surfaces 270, 272 and the humerus and the ulna.

In other embodiments of device 200, the cutting edges of the collar could be detachable so that they are replaced after becoming dull. The pins which hold the slider inside the head could be oriented 90° from the current embodiment. The slider could be moved so that advancing the screw increases offset, whereas the embodiment shown above reduces offset.

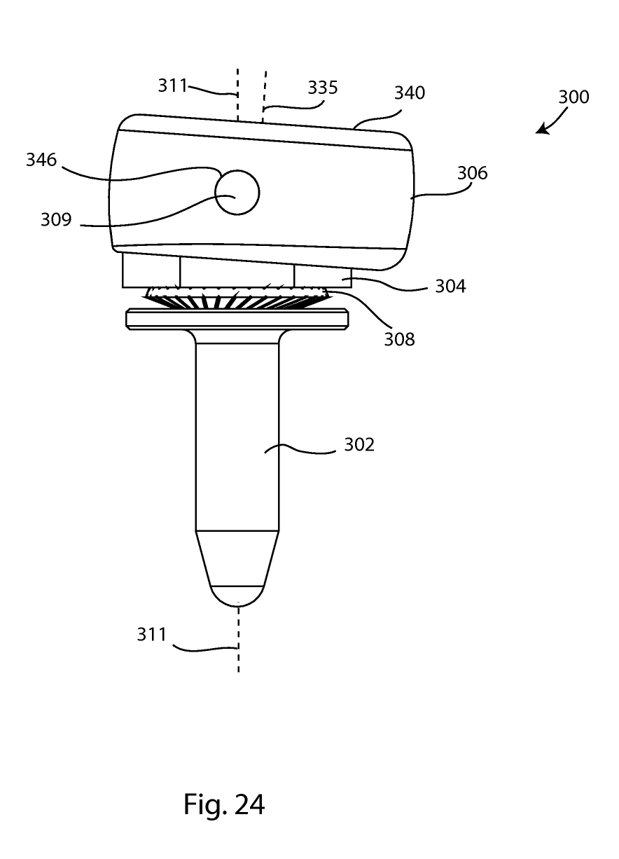

FIGS. 24-26 show a radial head trial device 300 which allows for adjusting the angular position of the trial head component. The device may include a stem, a head, a pin, a Belleville washer, and a sinusoidal washer.

In this application, a sinusoidal washer is defined as a washer with at least one of its two faces defined by projecting a complete sine wave around the perimeter of a cylinder, as though the sine wave had been drawn on a flat sheet which was then rolled to form the cylinder. The opposite face of the sinusoidal washer may be flat, in which case the washer has its maximum thickness at the local maximum of the sine wave, and its minimum thickness at the local minimum of the sine wave.

The components may be assembled together during manufacturing. The device can be inserted into the proximal opening of the radius after the native radial head has been removed and the proximal shaft has been progressively reamed. The stem may be made available in a range of diameters to suit different size patients. The head component is connected to the stem component with a pin allowing the head to pivot in a medial lateral direction around the pin. The angular adjustment of the head is made possible by a pre-compressed six sided sinusoidal washer that is compressed using a Belleville washer. As the sinusoidal washer is rotated the pivoting radial head will follow the ramp of the washer. The Belleville washer serves to compress the sinusoidal washer against the head component at all times to allow the radial head to maintain its adjusted position from the positive pressure applied by the washer-washer interaction on the radial head. A scale can be included on the bottom of the head to show the angulation of the head from the stem after trialing is complete. The head component can be made in different offsets and sizes.

Radial head trial device 300 includes the stem component 302, an intermediate component 304, and a head or cap component 306. In this embodiment, the intermediate component 304 is a sinusoidal washer which has an angled or slanted upper surface. The device 300 further includes a Belleville washer 308, and a pin 309.

The stem component 302 may include a shaft portion 310, a collar 312 and a cylindrical attachment portion 314. A stem channel 316 sized to receive pin 309 extends through the attachment portion 314. The stem component has a center longitudinal axis 311. The sinusoidal washer 304 is hexagonal and has a superior surface 320 and an inferior surface 322. The superior surface 320 is non-parallel, or angled or slanted relative to the inferior surface 322. The sinusoidal washer 304 is circumscribed by a peripheral wall 324 which extends between the superior and inferior surfaces 320, 322. The height of the peripheral wall varies according to the angle of the superior surface 320. Although a hexagonal washer is shown, the sinusoidal washer may have another shape including round, square, octagonal or pentagonal. The washer may also be described as a helical washer.

The cap component 306 is generally disc or puck shaped, and includes a superior end 330, an inferior end 332, and a peripheral wall 334 extending between the ends 330, 332. A first bearing surface 340, which may be dished, is formed on the superior end 330 and is shaped to articulate with a distal humerus. In the example shown, a cap center axis 335 which passes through the first bearing surface 340 is offset from and non-parallel with the center longitudinal axis 311. A second bearing surface 342 is formed on the peripheral wall 334 and may be shaped to articulate with a proximal ulna. A recess 344 extends inwardly from the inferior end 332, to receive the attachment portion 314 of the stem component. As seen in FIG. 26, the recess 344 is wider and taller than the attachment portion 314, to allow the head component 306 to pivot relative to the stem component 302 while attached to the stem component via the pin 309. A channel 346 extends transversely through the cap component 306 to receive the pin 309.

When device 300 is fully assembled, Belleville washer 308 may be threaded over attachment portion 314 to rest against collar 312. Sinusoidal washer 304 is also threaded over attachment portion 314 to rest against the Belleville washer 308. Cap component 306 is placed on the device with attachment portion 314 received in recess 344. Pin 309 may be passed into channel 346 and stem channel 316 to attach the cap component 306 to the stem component 314.

The Belleville washer 308 provides compressive force to urge the sinusoidal washer against the inferior end 332 of the cap component 306.

In a method of use, the assembled device 300 may be inserted into a prepared proximal radius. The sinusoidal washer 304 may be rotated relative to the rest of the device 300. In one embodiment an instrument such as a wrench is used to rotate washer 304, although the washer 304 may be rotated manually or by other suitable instruments. As the washer 304 is rotated, the angled superior surface 320 of the washer will act as a ramp to pivot the cap component 306 relative to the stem component 302 and change the angle of the cap component relative to the stem component. More specifically, the angle between the center axis 335 of the cap component and the center longitudinal axis 311 of the stem component will change, as will the angle between the articular bearing surface 340 and the center longitudinal axis 311. The cap component may be adjusted to be flat, such that the transverse plane of the cap is parallel to the transverse plane of the stem component, or it may be adjusted to be angled such that the transverse plane of the cap is non-parallel with the transverse plane of the stem component. Once a desired angle of the cap component 306 relative to the surrounding anatomy and the stem component 302 is reached, the angle may be determined and a radial head prosthesis may be selected which matches the angle of the trial device 300. Device 300 may allow selection of a prosthesis without substituting individual cap components of various angles, since the angle variation is built into the trialing device. The time for surgery may be reduced because the surgeon is not required to remove the trial and assemble alternative trial configurations. Device 300 may provide better comparison between angular positions than other radial head trials, and allows for an infinite resolution of angular positions when compared to non-adjustable trials.

It is appreciated that the Belleville washer and sinusodal washer interaction could be used on any device to locate and position a trial implant or instrument to ensure correct angular positioning of the device. For example, angular positioning for a wrist, hip, knee, shoulder, toe, finger, or spinal joint implant could be accomplished by the devices and methods disclosed herein.

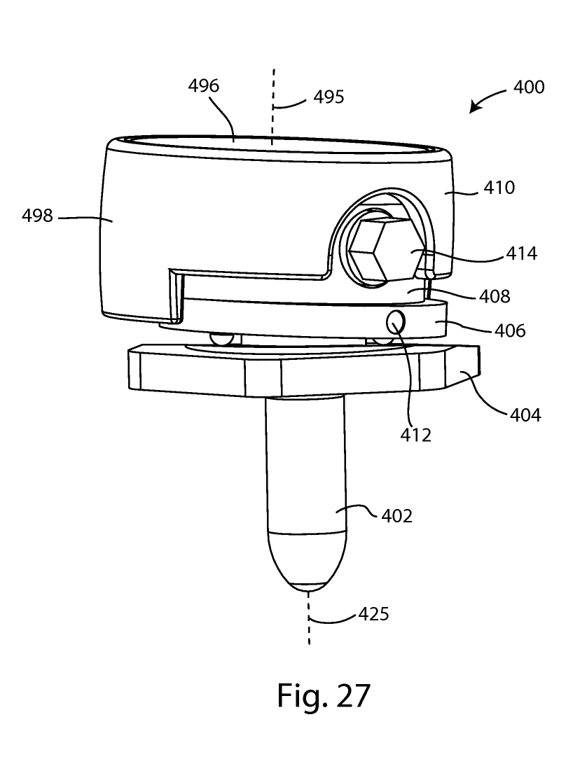

Some embodiments may include a combination of the offset and angular adjustments disclosed herein. For example, radial head trial device 400 includes the offset adjustability of device 200 with the angle adjustability of device 300, providing a radial head trial with both in situ offset adjustment and in situ angular adjustment. Referring to FIGS. 27-30B, radial head trial device 400 includes stem component 402, a sinusoidal washer 404, a washer 406, an intermediate component 408, and a radial head or cap component 410. A pin 412 pivotably connects the washer 406 to the stem component. A threaded pin 414 connects the intermediate component 408 to the stem component, allowing translation of the intermediate component and attached cap component relative to the stem component but preventing axial movement of the intermediate and cap components relative to the stem component. When sinusoidal washer 404 is rotated relative to the stem component 402, cams on washer 406 contacting sinusoidal washer 404 change the slope of the cap component 410 relative to the sinusoidal washer and the stem component 402. The cap component may be adjusted to be flat, such that the transverse plane of the cap is parallel to the transverse plane of the stem component, or it may be adjusted to be angled such that the transverse plane of the cap is non-parallel with the transverse plane of the stem component.

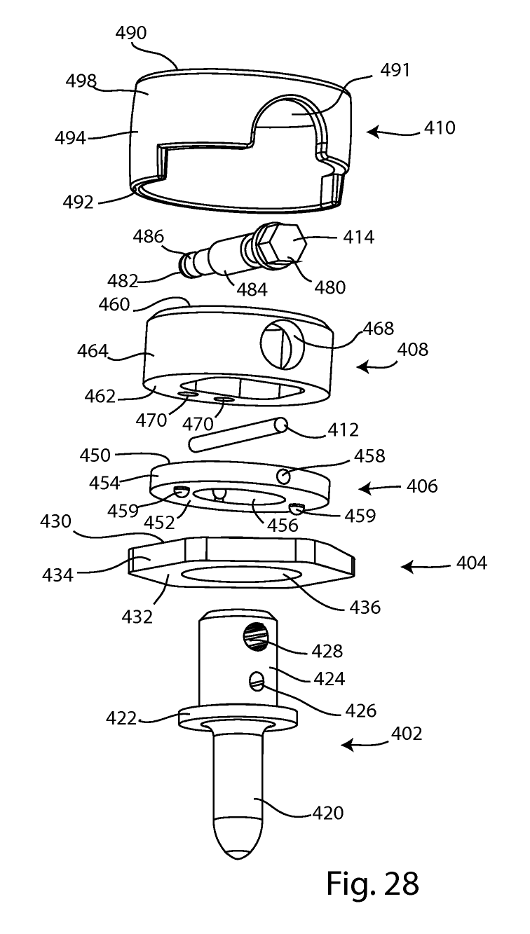

Referring to FIG. 28, stem component 402 comprises a shaft portion 420 and an attachment portion 424, with a flange 422 positioned between the shaft and attachment portions. A first stem bore 426 and a second stem bore 428 extend transversely through the attachment portion 424. Portions of either bore may be threaded. The stem component 402 has a center longitudinal stem axis 425.

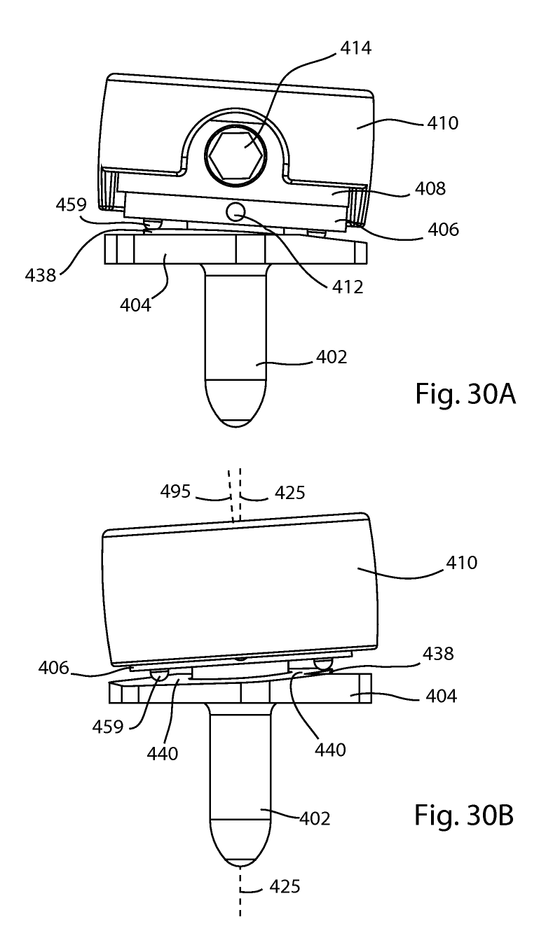

Referring to FIGS. 28, 30A and 30B, the sinusoidal washer 404 is hexagonal and has a superior surface 430 and an inferior surface 432. The superior surface 430 is non-parallel, or angled or slanted relative to the inferior surface 432. The sinusoidal washer 404 is circumscribed by a peripheral wall 434 which extends between the superior and inferior surfaces 320, 322. A washer bore 436 extends between the superior and inferior surfaces. The height of the peripheral wall varies according to the angle of the superior surface 430. Although a hexagonal washer is shown, the sinusoidal washer may have another shape including round, square, octagonal or pentagonal. A ramp 438 projects superiorly from a portion of the superior surface, and overhangs a portion of the washer bore 436. A helical sloped surface 440 occupies the superior side of the ramp 438 and a portion of the superior surface 430.

Washer 406 is circular and includes a superior surface 450, an inferior surface 452, a peripheral wall 454 extending between the inferior and superior surfaces, and a first washer bore 456 extending axially between the inferior and superior surfaces. A second washer bore 458 extends transversely across the washer, and is sized to receive pin 412. At least two cams 459 project inferiorly from the inferior surface 452. In the example shown, the cams 459 lie on an axis perpendicular to the axis of the second washer bore 458.

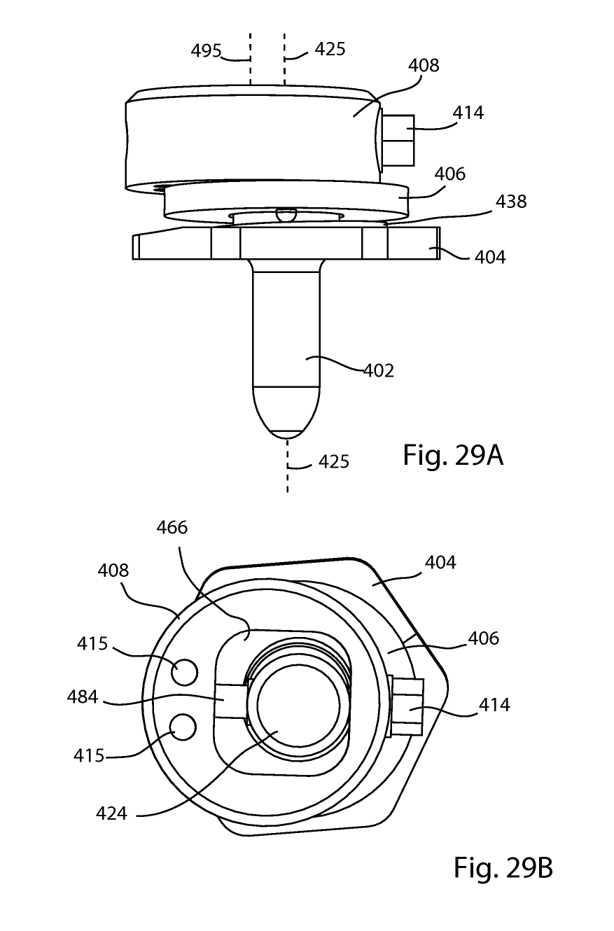

Referring to FIGS. 28-29B, intermediate component 408 is generally disc or puck-shaped, and includes a superior end 460, an inferior end 462, with a peripheral wall 464 extending between the superior and inferior ends. A bore 466 extends axially between the extending superior and inferior ends. A first pin bore 468 extends transversely across the component 408, and may have sections of varying diameters. Two second pin bores 470 extend axially through the component 408, offset from the bore 466. Threaded pin 414 includes a first head end 480, a second end 482, and a pin shaft 484 extending therebetween. A portion of the pin shaft 484 may be threaded. Adjacent the second end 482, at least one retention feature 486 is formed. In the embodiment shown, retention feature 486 is a groove circumscribing and recessed into the pin shaft 484.

Referring to FIGS. 27-28, cap component 410 includes a superior end 490, an inferior end 492, with a peripheral wall 494 extending between the superior and inferior ends. A proximal articular bearing surface 496 is formed on the superior end; the interior end 492 is open. Articular bearing surface 496 may be concave, or dished. A second articular bearing surface 498 is formed on the peripheral wall 494, and may be convex. A cutout 491 is formed in a portion of the peripheral wall 494, open to the interior end 492 and separated from the superior end 490. The cutout 491 provides access to pins 412, 414 when the device 400 is assembled. The cap component 410 includes a cap center axis 495.

Referring to FIGS. 29A and 29B, a subassembly of device 400 is shown in order to illustrate the offset adjustment mechanism. In the subassembly, the washer bore 436 of sinusoidal washer 404 is threaded over the attachment portion 424 of the stem component 402, so that a portion of ramp 438 is supported by the stem flange 422. Washer 406 is located superior to sinusoidal washer 404 and also encircles attachment portion 424. Pin 412 extends through second washer bore 458 and first stem bore 426, tethering washer 406 to stem component 402 while allowing pivoting of washer 406 about the longitudinal axis of the pin 412, as may be seen in FIG. 19B. Pin 412 also prohibits axial movement of washer 406 relative to stem component 402. Intermediate component 408 sits atop washer 406 with attachment portion 424 received in bore 466 and the inferior end 462 of component 408 flush with the superior surface 450 of washer 406. Bore 466 is wider than attachment portion 424, allowing space for translation of attachment portion 424 relative to, and within, intermediate component 408. Threaded pin 414 extends through first pin bore 468 and second stem bore 428. Threads on pin shaft 484 engage threads in second stem bore 428. Two retention pins 415 extend through second pin bores 470 and engage retention feature 486 on threaded pin 414 to capture the intermediate component 408 relative to the pin 414. When threaded pin 414 is rotated, the captured intermediate component 408 translates relative to attachment portion 424, along the longitudinal axis of the shaft 484 of threaded pin 414. When cap component 410 is included as in FIG. 27, this translation provides the offset adjustment of cap component 410 relative to the stem component 402, changing the distance between cap axis 495 and stem axis 425. In the example shown, up to 2 mm of offset is available through rotation of threaded pin 414. Other embodiments may provide more than 2 mm offset. Cap component 410 may be adjusted as described such that cap axis 495 is coaxial with stem axis 425, or it may be adjusted as described such that cap axis 495 is offset from stem axis 425. In some configurations, the cap axis 495 may be skewed from the stem axis 425.

Referring to FIGS. 30A and 30B, the angle of the cap component 410 may be adjusted relative to the stem component 402. Sinusoidal washer 404 is rotated, either manually or with a wrench or other instrument. As the sinusoidal washer 404 rotates, cams 459 on washer 406 contact helical sloped surface 440 at two contact points, and the angle of washer 406 relative to stem 402 changes. Intermediate component 408 and cap component 410 are carried by washer 406 and therefore also are angularly adjusted by the rotation of sinusoidal washer 404. In the example shown, rotation of the sinusoidal washer changes the slope between the contact points to provide angular adjustment along a continuum of 0°+/−5°. Other embodiments may include more than +/−5° of angular adjustment. Angle and offset adjustment may occur in any order.

Some embodiments may include a stem component having a shaft that is curved, or a shaft that is multi-segmented, having 2 or more angled segments. The outer peripheral surface of the shaft may be generally rounded or cylindrical, or may be multi-faceted. In cross-section, the shaft may be circular, square, rectangular, triangular, hexagonal, star-shaped, or another cross-sectional shape. Any of the devices disclosed herein may include markings to indicate size, offset, and/or angulation.

The present invention may be embodied in other specific forms without departing from its spirit or essential characteristics. It is appreciated that various features of the above-described examples can be mixed and matched to form a variety of other alternatives. Head components, stem components or intermediate components may be interchangeable in any of the embodiments set forth herein, as may the orientation of the head component relative to the stem component. For example, the meshed connection of prosthesis 1500 may be incorporated into the connections between the components of any of the other prosthesis embodiments. Trial head components, stem components, intermediate components and/or adjustment mechanisms may be interchangeable in any of the embodiments set forth herein, as may the orientation of the head component relative to the stem component. It is also contemplated that the embodiments disclosed herein may have application outside of the elbow joint; for example in wrist, shoulder, ankle, knee, or hip arthroplasty. As such, the described embodiments are to be considered in all respects only as illustrative and not restrictive.

It should be understood that the present system, kits, apparatuses, and methods are not intended to be limited to the particular forms disclosed. Rather, they are to cover all modifications, equivalents, and alternatives falling within the scope of the claims.

The claims are not to be interpreted as including means-plus- or step-plus-function limitations, unless such a limitation is explicitly recited in a given claim using the phrase(s) “means for” or “step for,” respectively.

The term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

1. A system comprising:
a head component comprising an articular surface and a first attachment feature opposite the articular surface, wherein the articular surface comprises a rotation axis centered in the articular surface, wherein the first attachment feature comprises a central longitudinal first axis which is other than coaxial with the rotation axis; and
a stem component coupled to the head component, the stem component comprising a second attachment feature and a shaft, wherein the second attachment feature comprises a central longitudinal second axis, wherein the shaft comprises a central longitudinal third axis which is other than coaxial with the second axis;
wherein the first attachment feature engages the second attachment feature to couple the head component to the stem component at any one of a plurality of rotational orientations of the head component about the second axis.

2. The system of claim 1, wherein the first attachment feature and the second attachment feature couple the head component to the stem component at any one of a plurality of discrete rotational orientations of the head component about the second axis.

3. The system of claim 1, wherein the first axis is parallel to the rotation axis and, wherein the first axis is offset from the rotation axis by a first distance, wherein the third axis is parallel to the second axis, wherein the third axis is offset from the second axis by a second distance.

4. The system of claim 3, wherein the first axis is coaxial with the second axis.

5. The system of claim 4, wherein the second distance is equal to the first distance.

6. The system of claim 1, wherein the head component comprises a peripheral wall extending between a first side and a second side opposite the first side, wherein the first side comprises the articular surface, wherein the second side comprises the first attachment feature, wherein the peripheral wall comprises a central longitudinal fourth axis, wherein the fourth axis has an orientation relative to the first axis which is selected from the group of orientations consisting of parallel, intersecting, and skew.

7. A system comprising:
a head comprising an articular surface, wherein the articular surface comprises a rotation axis centered in the articular surface; and
a stem coupled to the head by a connection mechanism, wherein the stem comprises a shaft, wherein the shaft comprises a central longitudinal shaft axis, wherein the connection mechanism comprises a first axis, wherein the connection mechanism provides a plurality of rotational orientations of the head relative to the stem about the first axis, wherein at least one of the rotation axis and the shaft axis is other than coaxial with the first axis.

8. The system of claim 7, wherein the connection mechanism couples the head to the stem at any one of a plurality of discrete rotational orientations of the head about the first axis.

9. The system of claim 7, wherein the first axis is parallel to the rotation axis and, wherein the first axis is offset from the rotation axis by a first distance, wherein the shaft axis is parallel to the first axis, wherein the shaft axis is offset from the first axis by a second distance.

10. The system of claim 9, wherein the second distance is equal to the first distance.

11. The system of claim 7, wherein the head comprises a peripheral wall extending between a first side and a second side opposite the first side, wherein the first side comprises the articular surface, wherein the second side comprises the connection mechanism, wherein the peripheral wall comprises a central longitudinal head axis, wherein the head axis has an orientation relative to the first axis which is selected from the group of orientations consisting of parallel, intersecting, and skew.

12. A system comprising:
a head component comprising an articular surface and a first attachment feature opposite the articular surface, wherein the articular surface comprises a rotation axis centered in the articular surface, wherein the first attachment feature comprises a central longitudinal first axis, wherein the first axis has an orientation relative to the rotation axis which is selected from the group of orientations consisting of parallel, intersecting, and skew; and
a stem component coupled to the head component, the stem component comprising a second attachment feature and a shaft, wherein the second attachment feature comprises a central longitudinal second axis, wherein the shaft comprises a central longitudinal third axis, wherein the third axis has an orientation relative to the second axis which is selected from the group of orientations consisting of parallel, intersecting, and skew;
wherein the first attachment feature and the second attachment feature couple the head component to the stem component at any one of a plurality of rotational orientations of the head component about the second axis.

13. The system of claim 12, wherein the first attachment feature and the second attachment feature couple the head component to the stem component at any one of a plurality of discrete rotational orientations of the head component about the second axis.

14. The system of claim 12, wherein the first axis is parallel to the rotation axis and, wherein the first axis is offset from the rotation axis by a first distance, wherein the third axis is parallel to the second axis, wherein the third axis is offset from the second axis by a second distance.

15. The system of claim 14, wherein the first axis is coaxial with the second axis.

16. The system of claim 15, wherein the second distance is equal to the first distance.

17. The system of claim 12, wherein the head component comprises a peripheral wall extending between a first side and a second side opposite the first side, wherein the first side comprises the articular surface, wherein the second side comprises the first attachment feature, wherein the peripheral wall comprises a central longitudinal fourth axis, wherein the fourth axis has an orientation relative to the first axis which is selected from the group of orientations consisting of parallel, intersecting, and skew.

18. The system of claim 17, wherein the fourth axis has an orientation relative to the rotation axis which is selected from the group of orientations consisting of parallel, intersecting, and skew.

19. The system of claim 12, wherein the first attachment feature and the second attachment feature couple the head component to the stem component at any one of an infinite number of discrete rotational orientations of the head component about the second axis.

20. The system of claim 12, comprising an intermediate component coupled to the head component and the stem component, the intermediate component comprising a third attachment feature and a fourth attachment feature, wherein the third attachment feature comprises a central longitudinal fifth axis, wherein the fourth attachment feature comprises a central longitudinal sixth axis, wherein the fifth axis has an orientation relative to the fourth axis which is selected from the group of orientations consisting of parallel, intersecting, and skew, wherein the first attachment feature engages the third attachment feature and the second attachment feature engages the fourth attachment feature to couple the head component to the stem component.
